(19) United States
(12) Patent Application Publication
(10) Pub. No.: US 2023/0257390 A1
(43) Pub. Date: Aug. 17, 2023

(54) COMPOUNDS WHICH INHIBIT RNA POLYMERASE AND THEIR USE

(71) Applicants: The Johns Hopkins University, Baltimore, MD (US); Lieber Institute, Inc., Baltimore, MD (US)

(72) Inventors: Hester Hui Liu, Cockeysville, MD (US); James C. Barrow, Arnold, MD (US); Marikki K. Laiho, Kauniainen (FI); Rajesh Kumar Nv, Baltimore, MD (US); Pablo de Leon, Baltimore, MD (US); Tony Dorado, Baltimore, MD (US); Asma Begum, Baltimore, MD (US); Wenjun Fan, Baltimore, MD (US); Gregory Stachelek, Baltimore, MD (US)

(21) Appl. No.: 18/024,421

(22) PCT Filed: Sep. 15, 2021
(86) PCT No.: PCT/US2021/050412

§ 371 Date: Mar. 2, 2023

Related U.S. Application Data
(60) Provisional application No. 63/078,723, filed on Sep. 15, 2020.

(51) Int. Cl. C07D 491/147; C07D 471/04

(52) U.S. Cl. CPC C07D 491/147; C07D 471/04

(57) ABSTRACT

This disclosure features chemical entities (e.g., a compound or a pharmaceutically acceptable salt, and/or hydrate, and/or cocrystal, and/or drug combination of the compound) that inhibit RNA polymerase I (Pol I). Said chemical entities are useful, e.g., for treating a condition, disease or disorder in which increased (e.g., excessive) Pol I activity contributes to the pathology and/or symptoms and/or progression of the condition, disease or disorder (e.g., cancer) in a subject (e.g., a human). This disclosure also features compositions containing the same as well as methods of using and making the same.

### CROSS REFERENCE TO RELATED APPLICATION

This application claims the benefit of U.S. Provisional Application Ser. No. 63/078,723, filed on Sep. 15, 2020, which is incorporated herein by reference in its entirety.

### BACKGROUND

Ribosomal (r) DNA is the most highly transcribed genomic region of the human genome and occurs in a dedicated subcellular compartment, the nucleolus. Transcription of rRNA is mediated by RNA polymerase I (Pol I) that transcribes the multicopy rDNA gene to a long 47S rRNA precursor. The 47S rRNA precursor is processed through multiple steps to the 18S, 5.8S and 28S mature rRNAs requisite for the assembly of the ribosomes. Pol I transcription is initiated by binding of a multisubunit preinitiation complex to rDNA promoter, which stochastically recruits the Pol I holocomplex. The Pol I holocomplex is composed of 14 subunits in eukaryotes, of which the subunits RPA194, RPA135 and RPA12 form the catalytically active site. Destabilization of the rDNA helix, or loss of the protein framework, will effectively stall transcription. The rate of rRNA transcription is tightly controlled by external signaling pathways that cause the assembly and binding of the preinitiation complex. Deregulation of rRNA synthesis is highly frequent in human cancers. This is due to activation of extracellular and intracellular signaling pathways and oncogenes such as Myc. Conversely, loss-of-function of tumor suppressors p53, pRB, ARF and PTEN lead to activation of Pol I transcription. Therefore, inhibitors of Pol I transcription may provide novel approaches toward cancer therapies.

12H-Benzo[g]pyrido[2,1-b]quinazoline-4-carboxamide, N-[2(dimethylamino)ethyl]-12-oxo (BMH-21) has been shown to exhibit a distinct mode of inhibition of Pol I compared to CX-5461 (see, e.g., U.S. Pat. No. 8,680,107). It is believed that BMH-21 intercalates with GC-rich rDNA, inhibits Pol I and causes proteasome-mediated degradation of RPA194. BMH-21 has also shown anticancer activity in NCI60 cancer cell lines and reduced tumor burden in mouse xenograft assays. WO 2015/143293, filed Mar. 20, 2015, discloses 12-oxo-12H-benzo[g]pyrido[2,1-b]quinazoline-4-carboxamide derivatives that inhibit Pol I.

### SUMMARY

This disclosure features chemical entities (e.g., a compound or a pharmaceutically acceptable salt thereof) that inhibit Pol I. Said chemical entities are useful, e.g., for treating a condition, disease or disorder in which increased (e.g., excessive) Pol I activity contributes to the pathology and/or symptoms and/or progression of the condition, disease or disorder (e.g., cancer) in a subject (e.g., a human). This disclosure also features compositions containing the same as well as methods of using and making the same.

In one aspect, this disclosure features compounds of Formula (I):

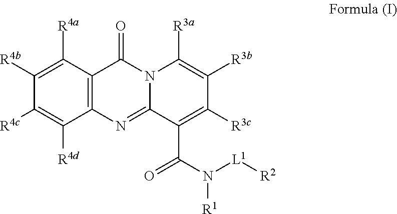

or a pharmaceutically acceptable salt thereof, wherein. R¹, L¹, R², R^{3a}, R^{3b}, R^{3c}, R^{4a}, R^{4b}, R^{4c}, and R^{4d} can be as defined elsewhere herein.

In another aspect, this disclosure features pharmaceutical compositions that include one or more compounds of Formula (I), and one or more pharmaceutically acceptable carriers.

In a further aspect, this disclosure features methods for activating upstream p53 pathways in a mammalian cell that include contacting a cell or population of cells with a compound of Formula (I), or a pharmaceutically acceptable salt thereof, or a pharmaceutical composition containing the same.

In still another aspect, this disclosure features methods for modulating RNA Pol I activity in a mammalian cell that include contacting a cell or population of cells with a compound of Formula (I), or a pharmaceutically acceptable salt thereof, or a pharmaceutical composition containing the same

In yet a further aspect, this disclosure features methods for treating cancer in a subject in need of such treatment that includes administering to the subject a compound of Formula (I), a pharmaceutically acceptable salt thereof, or a pharmaceutical composition containing the same.

In another aspect, this disclosure features methods for treating cancer in a subject in need thereof that includes administering to the subject a compound of Formula (I), or a pharmaceutically acceptable salt thereof, or a pharmaceutical composition containing the same, and at least one additional therapeutic agent.

In one aspect, this disclosure features methods for treating an autoimmune disease or disorder in a subject in need thereof that includes administering to the subject a compound of Formula (I), or a pharmaceutically acceptable salt thereof, or a pharmaceutical composition containing the same.

In another aspect, this disclosure features methods for treating a condition associated with inflammation or pain in a subject in need thereof that includes administering to the subject a compound of Formula (I), or a pharmaceutically acceptable salt thereof, or a pharmaceutical composition containing the same.

In certain of the foregoing embodiments, the methods further include administering to the subject one or more additional therapeutic agents.

### DETAILED DESCRIPTION

This disclosure features chemical entities (e.g., a compound or a pharmaceutically acceptable salt thereof) that inhibit Pol I. Said chemical entities are useful, e.g., for treating a condition, disease or disorder in which increased (e.g., excessive) Pol I activity contributes to the pathology and/or symptoms and/or progression of the condition, disease or disorder (e.g., cancer) in a subject (e.g., a human). This disclosure also features compositions containing the same as well as methods of using and making the same.

In one aspect, the present disclosure features compounds of Formula (I):

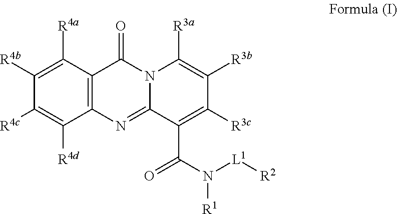

or a pharmaceutically acceptable salt thereof, wherein:

R¹ is H or C₁₋₃ alkyl;

R² is selected from the group consisting of:

(a) —NR⁶R⁷, wherein R⁶ and R⁷ are independently selected from the group consisting of: H and C₁₋₆ alkyl which is optionally substituted with from 1-6 R^{a}; and

(b) -heterocyclyl including from 4-12 ring atoms, wherein from 1-3 ring atoms are ring heteroatoms each independently selected from the group consisting of N, NH, N(R^{d}), O, and S(O)₀₋₂, wherein the heterocyclyl is optionally substituted with from 1-6 R^{b};

L¹ is a bond or C₁₋₆ alkylene which is optionally substituted with from 1-6 R^{c}, provided that when L¹ is a bond, then R² is heterocyclyl that is attached to L¹ via a ring carbon atom;

R^{3a}, R^{3b}, R^{3c}, R^{4a}, R^{4b}, R^{4c}, and R^{4d} are each independently selected from the group consisting of: H, halo, C₁₋₆ alkyl, C₁₋₆ haloalkyl, C₁₋₆ alkoxy, C₁₋₆ haloalkoxy, OH, C₃₋₆ cycloalkyl, and C₆₋₁₀ aryl; or

two of the variables R^{4a}, R^{4b}, R^{4c}, and R^{4d} on adjacent ring carbon atoms, taken together with the ring carbon atoms to which each is attached, forms a partially unsaturated ring including from 4-8 ring atoms, wherein from 0-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b};

each occurrence of R^{a} and R^{c} is independently selected from the group consisting of: —OH; -halo; —NR′R″; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;

each occurrence of R^{b} is independently selected from the group consisting of: C₁₋₆ alkyl optionally substituted with —OH, C₁₋₄ alkoxy, or C₁₋₄ haloalkoxy; C₁₋₆ haloalkyl; oxo; —OH; -halo; —NR′R″; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;

each occurrence of R^{d} is independently C₁₋₆ alkyl; —C(O)(C₁₋₄ alkyl); or —C(O)O(C₁₋₄ alkyl); and

each occurrence of R′ and R″ is independently H or C₁₋₃ alkyl.

As used herein, the term “halo” refers to fluoro (F), chloro (Cl), bromo (Br), or iodo (I).

As used herein, the term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

As used herein, the term “haloalkyl” refers to an alkyl, in which one or more hydrogen atoms is/are replaced with an independently selected halo.

As used herein, the term “alkoxy” refers to an —O-alkyl radical (e.g., —OCH₃). Accordingly, the term “haloalkoxy” refers to an —O-haloalkyl radical (e.g., —OCF₃).

As used herein, the term “alkylene” refers to a divalent alkyl (e.g., —CH₂—).

As used herein, the term “heterocyclyl” refers to a mono-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S(O)₀₋₂ (e.g., carbon atoms and 1-3, 1-6, or 1-9 heteroatoms of N, O, or S(O)₀₋₂ if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

Included within the compounds of the present disclosure are the tautomeric forms of the disclosed compounds, isomeric forms including diastereoisomers, and the pharmaceutically acceptable salts thereof.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)-, and individual isomers are encompassed within the scope of the disclosure. The compounds of the present disclosure do not include those which are known in art to be too unstable to synthesize and/or isolate. The disclosure is meant to include compounds in racemic and optically pure forms. Optically active (R)- and (S)-, isomers may be prepared using chiral synthons or chiral reagents as disclosed herein, or resolved using conventional techniques. When the compounds described herein contain olefinic bonds or other centers of geometric asymmetry, and unless specified otherwise, it is intended that the compounds include both E and Z geometric isomers.

In some embodiments, from 1-3 (e.g., 1, 2, or 3) of R^{4a}, R^{4b}, R^{4c}, and R^{4d} is other than H.

In some embodiments, R^{4b} and R^{4c} taken together with the ring carbon atoms to which each is attached, forms a partially unsaturated ring including from 4-8 ring atoms, wherein from 0-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b}.

In certain embodiments, R^{4b} and R^{4c} taken together with the ring carbon atoms to which each is attached, forms a partially unsaturated ring including from 4-6 ring atoms, wherein from 1-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b}.

As non-limiting examples of the foregoing embodiments, R^{4b} and R^{4c} taken together with the ring carbon atoms to which each is attached can form:

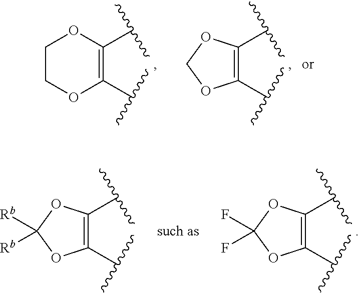

In certain embodiments, R^{4b} and R^{4c} taken together with the ring carbon atoms to which each is attached, forms a partially unsaturated ring including from 4-8 ring atoms, wherein the partially unsaturated ring does not include a ring heteroatom, and wherein the partially unsaturated ring is optionally substituted with from 1-3 R^{b}.

As a non-limiting example of the foregoing embodiments, R^{4b} and R^{4c} taken together with the carbon atoms to which each is attached can form.

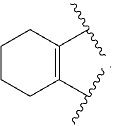 [SMILES: *C1=C(*)CCCC1]

In some embodiments, R^{4b} and R^{4c} are independently C₁₋₆ alkyl, C₁₋₆ haloalkyl, C₃₋₆ cycloalkyl, or C₆₋₁₀ aryl.

In certain embodiments, R^{4b} and R^{4c} are independently selected C₁₋₃ alkyl, such as methyl.

In some embodiments, R^{4a} and R^{4d} are H. In some embodiments when R^{4b} and R^{4c} are as defined supra, R^{4a} and R^{4d} are H.

In some embodiments, R^{4c} and R^{4d} taken together with the ring carbon atoms to which each is attached, forms a partially unsaturated ring including from 4-8 ring atoms, wherein from 0-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b}.

As a non-limiting example of the foregoing embodiments, R^{4c} and R^{4d} taken together with the carbon atoms to which each is attached can form

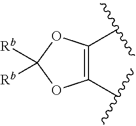

such as

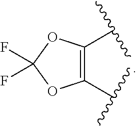 [SMILES: *C1=C(*)OC(F)(F)O1]

In some embodiments, R^{4c} and R^{4d} are independently C₁₋₆ alkyl, C₁₋₆ haloalkyl, C₃₋₆ cycloalkyl, or C₆₋₁₀ aryl.

In certain embodiments, R^{4c} and R^{4d} are independently selected C₁₋₃ alkyl, such as methyl.

In some embodiments, R^{4c} is C₁₋₆ alkyl, C₁₋₆ haloalkyl, C₃₋₆ cycloalkyl, or C₆₋₁₀ aryl; and R^{4d} is H.

In certain embodiments, R^{4c} is C₁₋₃ alkyl, such as methyl; and R^{4d} is H.

In certain embodiments, R^{4c} is phenyl; and R^{4d} is H.

In some embodiments, R^{4d} is C₁₋₆ alkyl, C₁₋₆ haloalkyl, C₃₋₆ cycloalkyl, or C₆₋₁₀ aryl; and R^{4c} is H.

In certain embodiments, R^{4d} is C₁₋₃ alkyl, such as methyl; and R^{4c} is H.

In certain embodiments, R^{4d} is phenyl; and R^{4c} is H.

In some embodiments, R^{4a} and R^{4b} are H. In certain embodiments when R^{4c} and R^{4d} are as defined supra, R^{4a} and R^{4b} are H.

In some embodiments, L¹ is C₁₋₆ alkylene optionally substituted with from 1-6 R^{c}.

In certain embodiments, L is a linear C₁₄ alkylene, optionally substituted with from 1-6 R^{c}.

As a non-limiting example of the foregoing embodiments, L¹ can be unsubstituted —CH₂CH₂—.

In some embodiments, L¹ is a branched C₃₋₄ alkylene optionally substituted with from 1-6 R^{c}.

As a non-limiting example of the foregoing embodiments, L¹ can be

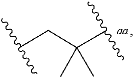

wherein aa is the point of attachment to R².

In some embodiments, R² is NR⁶R⁷.

In certain of these embodiments, R² is selected from the group consisting of: NH(C₁₋₃ alkyl) and N(C₁₋₃ alkyl)₂, such as wherein R² is —NMe₂.

In some embodiments, R² is heterocyclyl including from 4-12 ring atoms, wherein from 1-3 ring atoms are ring heteroatoms each independently selected from the group consisting of N, NH, N(R^{d}), O, and S(O)₀₋₂, wherein the heterocyclyl is optionally substituted with from 1-6 R^{b}.

In certain embodiments, R² is heterocyclyl including from 4-8 such as 4-6 ring atoms, wherein from 1-2 ring atoms are ring heteroatoms each independently selected from the group consisting of N, NH, N(R^{d}), O, and S(O)₀₋₂, wherein the heterocyclyl is optionally substituted with from 1-6 R^{b}.

As non-limiting examples of the foregoing embodiments, R² can be selected from the group consisting of:

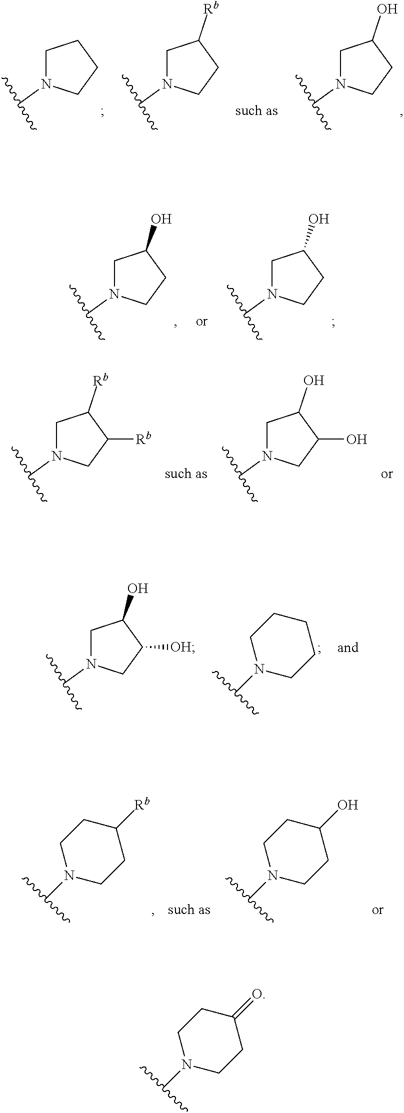

In some embodiments, R¹ is H.

In some embodiments, R^{3a}, R^{3b}, and R^{3c} are H.

In certain embodiments, the compound of Formula (I) is a compound of Formulae (I-a) or (I-b):

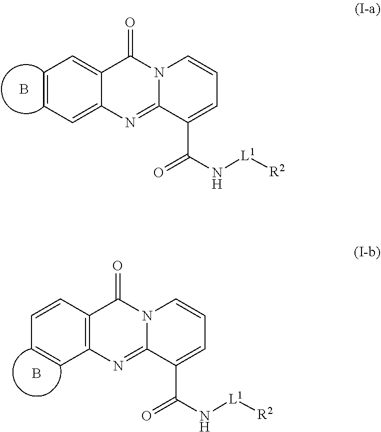

or a pharmaceutically acceptable salt thereof, wherein:

Ring B is selected from the group consisting of:

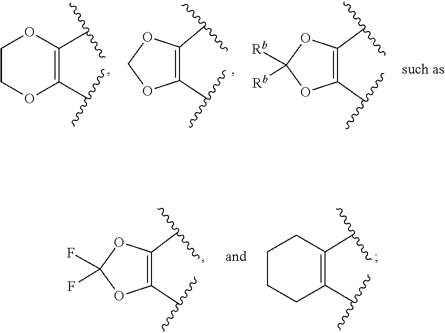

R² is selected from the group consisting of:
(i) heterocyclyl including from 4-8 such as 4-6 ring atoms, wherein from 1-2 ring atoms are ring heteroatoms each independently selected from the group consisting of N, NH, N(R^{d}), O, and S(O)₀₋₂, wherein the heterocyclyl is optionally substituted with from 1-6 R^{b}; and
(ii) NH(C₁₋₃ alkyl) or N(C₁₋₃ alkyl)₂.

In certain embodiments of Formulae (I-a) or (I-b), Ring B is

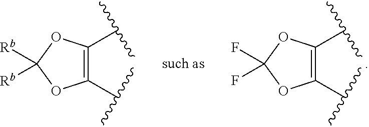

In certain embodiments of Formulae (I-a) or (I-b), Ring B is

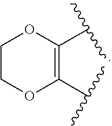 [SMILES: *C1=C(*)OCCO1]

In certain embodiments of Formulae (I-a) or (I-b), Ring B is

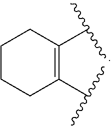 [SMILES: *C1=C(*)CCCC1]

In certain embodiments, the compound of Formula (I) is a compound of Formulae (I-c), (I-d), (I-e), or (I-f):

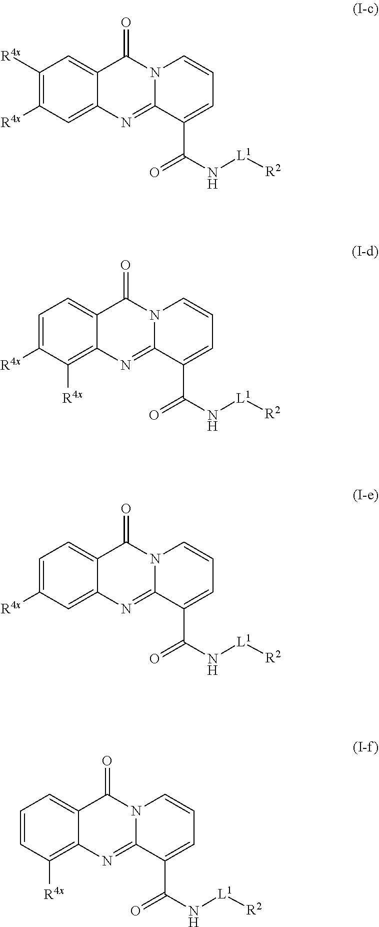

or a pharmaceutically acceptable salt thereof, wherein:

each R^{4x} is selected from the group consisting of: C₁₋₆ alkyl, C₁₋₆ haloalkyl, C₃₋₆ cycloalkyl, or C₆₋₁₀ aryl; and

R² is selected from the group consisting of:
(i) heterocyclyl including from 4-8 such as 4-6 ring atoms, wherein from 1-2 ring atoms are ring heteroatoms each independently selected from the group consisting of N, NH, N(R^{d}), O, and S(O)₀₋₂, wherein the heterocyclyl is optionally substituted with from 1-6 R^{b}; and
(ii) NH(C₁₋₃ alkyl) or N(C₁₋₃ alkyl)₂.

In certain embodiments of Formulae (I-c), (I-d), (I-e), or (I-f), each R^{4x} is independently C₁₋₃ alkyl, such as methyl.

In certain embodiments of Formulae (I-c), (I-d), (I-e), or (I-f), each R^{4x} is phenyl.

In certain embodiments of Formulae (I-a), (I-b), (I-c), (I-d), (I-e), or (I-f), L is a linear C₁₋₄ alkylene, optionally substituted with from 1-6 R^{c}. For example, L¹ can be —CH₂CH₂—.

In certain embodiments of Formulae (I-a), (I-b), (I-c), (I-d), (I-e), or (I-f), L¹ is a branched C₃₋₄ alkylene optionally substituted with from 1-6 R^{c}, such as wherein L¹ is

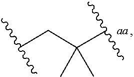

wherein aa is the point of attachment to R².

In certain embodiments of Formulae (I-a), (I-b), (I-c), (I-d), (I-e), or (I-f), R² is N(C₁₋₃ alkyl)₂, such as wherein R² is —NMe₂.

In certain embodiments of Formulae (I-a), (I-b), (I-c), (I-d), (I-e), or (I-f), R² is selected from the group consisting of:

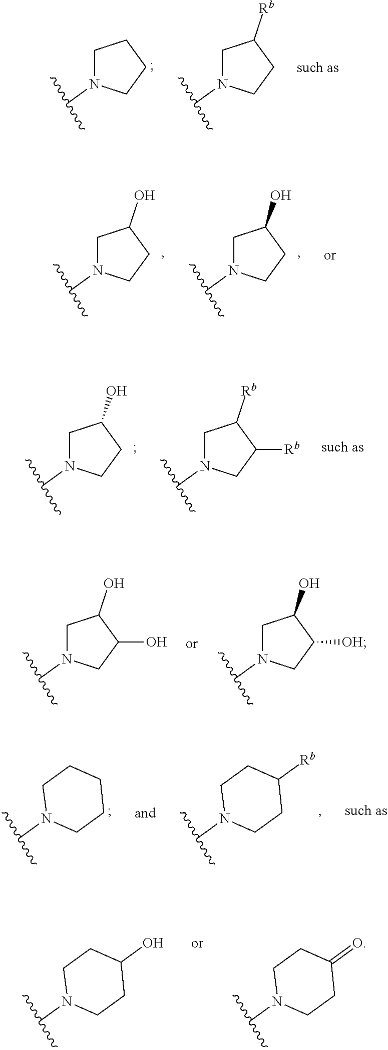

In certain embodiments, the compound of Formula (I) is selected from the group consisting of the compounds in Table C1, or a pharmaceutically acceptable salt thereof.

TABLE C1

| Compound | |
|---|---|
| No. | Structure |
| 101 | 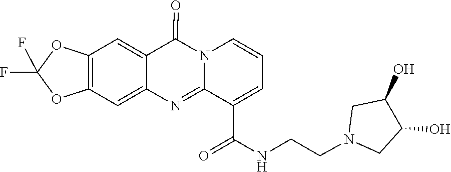 [SMILES: O=C(NCCN1C[C@@H](O)[C@H](O)C1)c1cccn2c(=O)c3cc4c(cc3nc12)OC(F)(F)O4] |
| 102 | 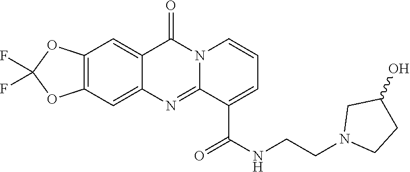 [SMILES: O=C(NCCN1CCC(O)C1)c1cccn2c(=O)c3cc4c(cc3nc12)OC(F)(F)O4] |
| 103 | 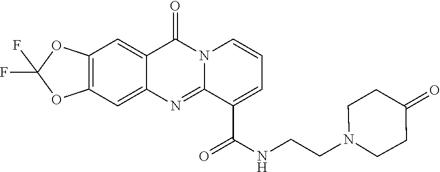 [SMILES: O=C1CCN(CCNC(=O)c2cccn3c(=O)c4cc5c(cc4nc23)OC(F)(F)O5)CC1] |
| 104 | 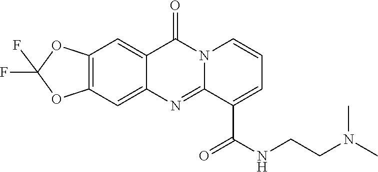 [SMILES: CN(C)CCNC(=O)c1cccn2c(=O)c3cc4c(cc3nc12)OC(F)(F)O4] |
| 105 | 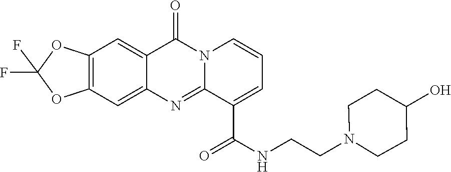 [SMILES: O=C(NCCN1CCC(O)CC1)c1cccn2c(=O)c3cc4c(cc3nc12)OC(F)(F)O4] |
| 106 | 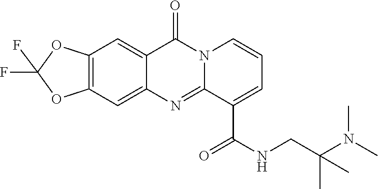 [SMILES: CN(C)C(C)(C)CNC(=O)c1cccn2c(=O)c3cc4c(cc3nc12)OC(F)(F)O4] |
| 107 | 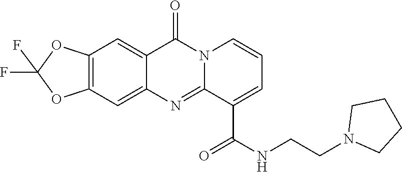 [SMILES: O=C(NCCN1CCCC1)c1cccn2c(=O)c3cc4c(cc3nc12)OC(F)(F)O4] |
| 108 | 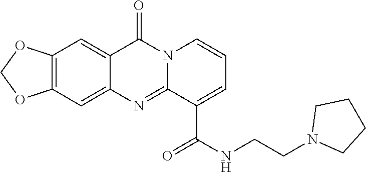 [SMILES: O=C(NCCN1CCCC1)c1cccn2c(=O)c3cc4c(cc3nc12)OCO4] |
| 109 | 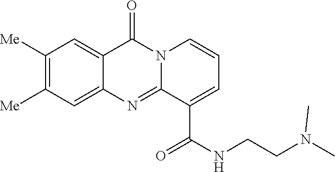 [SMILES: Cc1cc2nc3c(C(=O)NCCN(C)C)cccn3c(=O)c2cc1C] |
| 110 | 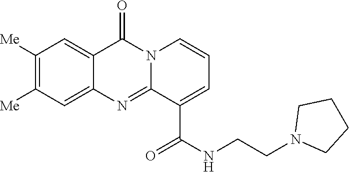 [SMILES: Cc1cc2nc3c(C(=O)NCCN4CCCC4)cccn3c(=O)c2cc1C] |
| 111 | 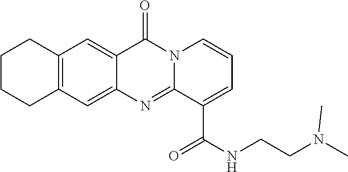 [SMILES: CN(C)CCNC(=O)c1cccn2c(=O)c3cc4c(cc3nc12)CCCC4] |
| 112 | 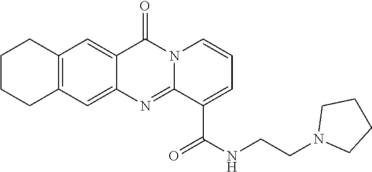 [SMILES: O=C(NCCN1CCCC1)c1cccn2c(=O)c3cc4c(cc3nc12)CCCC4] |
| 113 | 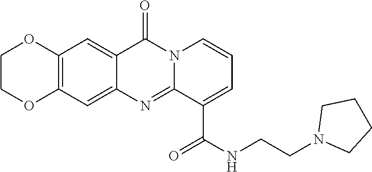 [SMILES: O=C(NCCN1CCCC1)c1cccn2c(=O)c3cc4c(cc3nc12)OCCO4] |
| 114 | 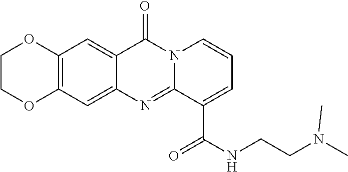 [SMILES: CN(C)CCNC(=O)c1cccn2c(=O)c3cc4c(cc3nc12)OCCO4] |
| 115 | 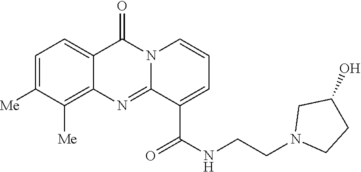 [SMILES: Cc1ccc2c(=O)n3cccc(C(=O)NCCN4CC[C@@H](O)C4)c3nc2c1C] |
| 116 | 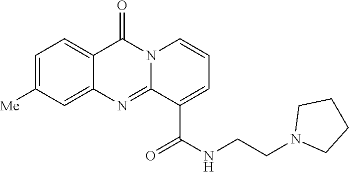 [SMILES: Cc1ccc2c(=O)n3cccc(C(=O)NCCN4CCCC4)c3nc2c1] |
| 117 | 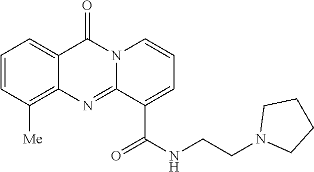 [SMILES: Cc1cccc2c(=O)n3cccc(C(=O)NCCN4CCCC4)c3nc12] |
| 118 | 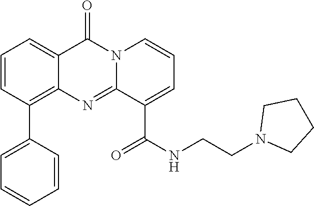 [SMILES: O=C(NCCN1CCCC1)c1cccn2c(=O)c3cccc(-c4ccccc4)c3nc12] |
| 119 | 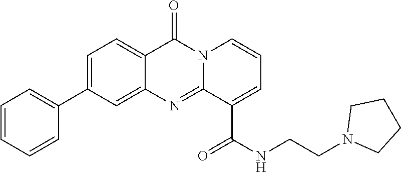 [SMILES: O=C(NCCN1CCCC1)c1cccn2c(=O)c3ccc(-c4ccccc4)cc3nc12] |
| 120 | 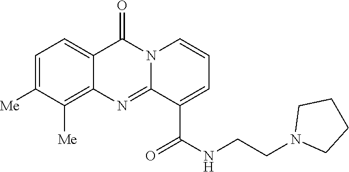 [SMILES: Cc1ccc2c(=O)n3cccc(C(=O)NCCN4CCCC4)c3nc2c1C] |
| 121 | 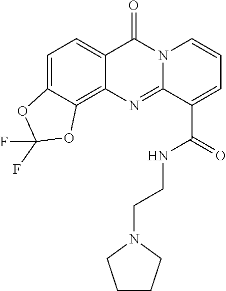 [SMILES: O=C(NCCN1CCCC1)c1cccn2c(=O)c3ccc4c(c3nc12)OC(F)(F)O4] |

The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the disclosure include, for example, acid addition salts which may, for example, be formed by mixing a solution of the compound according to the disclosure with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present disclosure.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present disclosure should be pharmaceutically acceptable salts. Other salts may, however, be useful in the preparation of the compounds according to the disclosure or of their pharmaceutically acceptable salts.

In addition, embodiments of the disclosure include hydrates of the compounds of the present disclosure. The term “hydrate” includes but is not limited to hemihydrate, monohydrate, dihydrate, trihydrate and the like. Hydrates of the compounds of the present disclosure may be prepared by contacting the compounds with water under suitable conditions to produce the hydrate of choice.

In one aspect, the present disclosure provides pharmaceutical compositions comprising the compounds of Formula (I), or their salts, solvates, or stereoisomers thereof, and a pharmaceutically acceptable carrier.

Embodiments of the disclosure also include a process for preparing pharmaceutical products comprising the compounds. The term “pharmaceutical product” means a composition suitable for pharmaceutical use (pharmaceutical composition), as defined herein. Pharmaceutical compositions formulated for particular applications comprising the compounds of the present disclosure are also part of this disclosure, and are to be considered an embodiment thereof.

As such, in another aspect, the present disclosure provides a pharmaceutical composition comprising the compound of Formula (I) and a pharmaceutically acceptable carrier. In some embodiments, the pharmaceutical composition can further comprise at least one additional therapeutic agent.

In another aspect, the present disclosure provides a pharmaceutical composition comprising a compound of Formula (I) and a pharmaceutically acceptable carrier, in an effective amount, for use as a medicament, e.g., for use in inhibiting RNA Pol I in a mammalian cell or population of cells, or for use in treating cancer in a subject.

With respect to pharmaceutical compositions described herein, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. Examples of the pharmaceutically acceptable carriers include soluble carriers such as known buffers which can be physiologically acceptable (e.g., phosphate buffer) as well as solid compositions such as solid-state carriers or latex beads. It is preferred that the pharmaceutically acceptable carrier be one which is chemically inert to the active agent(s), and one which has little or no detrimental side effects or toxicity under the conditions of use.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, in an embodiment, the compounds of the present disclosure may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the disclosure. The following formulations for parenteral, subcutaneous, intravenous, intramuscular, intraarterial, intrathecal and interperitoneal administration are exemplary, and are in no way limiting. More than one route can be used to administer the compounds, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-Q-aminopropionates, and 2-alkyl-imidazoline quaternary ammonium salts, and (e) mixtures thereof.

The parenteral formulations will typically contain from about 0.5% to about 25% by weight of the compounds in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the disclosure. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

In some embodiments, the chemical entities described herein or a pharmaceutical composition thereof are suitable for local and topical administration to the eye (e.g., eye drops). Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, the chemical entities described herein or a pharmaceutical composition thereof are suitable for local and topical administration to skin (e.g., ointments and creams). Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

For purposes of the disclosure, the amount or dose of the compounds, salts, solvates, or stereoisomers of any one the compounds of Formula (I), as set forth above, administered should be sufficient to effect, e.g., a therapeutic or prophylactic response, in the subject over a reasonable time frame. The dose will be determined by the efficacy of the particular compound and the condition of a human, as well as the body weight of a human to be treated.

It is understood by those of ordinary skill, that the compounds of the present disclosure are inhibitors of RNA polymerase I through one or more mechanisms of action. Without being limited to any particular theory, the compounds of the present disclosure can inhibit RNA Pol I by intercalation of the nucleic acids at G-C rich regions which block the polymerase activity.

One of ordinary skill in the art understands that p53 is a highly responsive molecule to cellular stress and DNA damage, and implicated in diverse diseases like cancer, ischemia, neuronal disorders, inflammation and also during physiological processes like in normal cellular metabolism, development and aging. Thus, the compounds of the present disclosure are useful in prevention or treatment of diseases involving the p53 pathways.

Therefore, in another aspect, the present disclosure provides the use of the compounds or the pharmaceutical compositions disclosed herein in an amount effective for activating upstream p53 pathways in a mammalian cell comprising contacting a cell or population of cells with a compound of Formula (I). Accordingly, in one aspect, provided herein is a method for activating upstream p53 pathways in a mammalian cell comprising contacting a cell or population of cells with a compound of Formula (I), a pharmaceutically acceptable salt thereof, or a pharmaceutical composition thereof. In certain embodiments, the step of contacting is carried out in vitro. In certain embodiments, the step of contacting is carried out in vivo.

In another aspect, the present disclosure provides the use of the compounds or the pharmaceutical compositions disclosed herein in an amount effective for modulating RNA Pol I activity in a mammalian cell comprising contacting a cell or population of cells with a compound of Formula (I). Accordingly, in one aspect, the disclosure provides a method for modulating RNA Pol I activity in a mammalian cell comprising contacting a cell or population of cells with a compound of Formula (I), a pharmaceutically acceptable salt thereof, or a pharmaceutical composition thereof. In certain embodiments, the step of contacting is carried out in vitro. In certain embodiments, the step of contacting is carried out in vivo.

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies. In some embodiments, the one or more additional cancer therapies is chemotherapy, which can include administering one or more additional chemotherapeutic agents.

In another aspect, the present disclosure provides the use of the compounds or the pharmaceutical compositions disclosed herein in an amount effective for treating a condition, disease or disorder in which increased (e.g., excessive) Pol I activity contributes to the pathology and/or symptoms and/or progression of the condition, disease or disorder in a subject comprising administering to the subject a pharmaceutical composition comprising a compound of Formula (I). In some embodiments, the use comprises administering to the subject at least one additional therapeutic agent.

Accordingly, in one aspect, the present disclosure provides a method for treating a condition, disease or disorder in which increased (e.g., excessive) Pol I activity contributes to the pathology and/or symptoms and/or progression of the condition, disease or disorder in a subject comprising administering to the subject a compound of Formula (I), a pharmaceutically acceptable salt thereof, or a pharmaceutical composition thereof. In some embodiments, the method further comprises administering to the subject at least one additional therapeutic agent.

In some embodiments, the present disclosure provides the use of the compounds or the pharmaceutical compositions disclosed herein in an amount effective for treating cancer or a hyperproliferative disease in a subject comprising administering to the subject a pharmaceutical composition comprising a compound of Formula (I). In some embodiments, the use comprises administering to the subject at least one additional therapeutic agent.

Accordingly, in some embodiments, the disclosure provides a method for treating cancer or a hyperproliferative disease in a subject comprising administering to the subject a compound of Formula (I), a pharmaceutically acceptable salt thereof, or a pharmaceutical composition thereof. In certain of these embodiments, the method further comprises administering to the subject at least one additional therapeutic agent.

In some embodiments, the present disclosure provides the use of the compounds or the pharmaceutical compositions disclosed herein in an amount effective for an autoimmune disease or disorder in a subject comprising administering to the subject a pharmaceutical composition comprising a compound of Formula (I). In some embodiments, the use comprises administering to the subject at least one additional therapeutic agent.

Accordingly, in some embodiments, the disclosure provides a method for treating an autoimmune disease or disorder in a subject comprising administering to the subject a compound of Formula (I), a pharmaceutically acceptable salt thereof, or a pharmaceutical composition thereof. In certain of these embodiments, the method further comprises administering to the subject at least one additional therapeutic agent.

In some embodiments, the present disclosure provides the use of the compounds or the pharmaceutical compositions disclosed herein in an amount effective for the treatment of a condition associated with inflammation or pain in a subject comprising administering to the subject a pharmaceutical composition comprising a compound of Formula (I). In some embodiments, the use comprises administering to the subject at least one additional therapeutic agent.

Accordingly, in some embodiments, the disclosure provides a method for treating of a condition associated with inflammation or pain in a subject comprising administering to the subject a compound of Formula (I), a pharmaceutically acceptable salt thereof, or a pharmaceutical composition thereof. In certain of these embodiments, the method further comprises administering to the subject at least one additional therapeutic agent.

In another aspect, the present disclosure provides a method for modulating angiogenesis in a subject, comprising administering to the subject a compound of Formula (I), a pharmaceutically acceptable salt thereof, or a pharmaceutical composition thereof. In certain of these embodiments, the method further comprises administering to the subject at least one additional therapeutic agent.

The dose of the compounds, salts, solvates, or stereoisomers of any one the compounds of Formula (I), as set forth above, of the present disclosure also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated. By way of example, and not intending to limit the disclosure, the dose of the compound can be about 0.001 to about 1000 mg/kg body weight of the subject being treated/day, from about 0.01 to about 100 mg/kg body weight/day, or from about 1 mg to about 100 mg/kg body weight/day. In some embodiments the dosage of the compound can be in the range of about 0.1 μM to about 100 μM, preferably about 1 μM to about 50 μM.

Alternatively, the compounds of the present disclosure can be modified into a depot form, such that the manner in which the compound is released into the body to which it is administered is controlled with respect to time and location within the body (see, for example, U.S. Pat. No. 4,450,150). Depot forms of compounds can be, for example, an implantable composition comprising the compound and a porous or non-porous material, such as a polymer, wherein the compound is encapsulated by or diffused throughout the material and/or degradation of the non-porous material. The depot is then implanted into the desired location within the body and the compounds are released from the implant at a predetermined rate.

In some embodiments, the compounds of the present disclosure provided herein can be controlled release compositions, i.e., compositions in which the one or more compounds are released over a period of time after administration. Controlled or sustained release compositions include formulation in lipophilic depots (e.g., fatty acids, waxes, oils). In another embodiment the composition is an immediate release composition, i.e., a composition in which all, or substantially all of the compound, is released immediately after administration.

In some embodiments, the compounds of the present disclosure can be delivered in a controlled release system. For example, the agent may be administered using intravenous infusion, an implantable osmotic pump, a transdermal patch, or other modes of administration. In an embodiment, a pump may be used. In some embodiments, polymeric materials can be used. In yet another embodiment, a controlled release system can be placed in proximity to the therapeutic target, i.e., the brain, thus requiring only a fraction of the systemic dose (see, e.g., Design of Controlled Release Drug Delivery Systems, Xiaoling Li and Bhaskara R. Jasti eds. (McGraw-Hill, 2006)).

The compounds included in the pharmaceutical compositions of the present disclosure may also include incorporation of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In accordance with the present disclosure, the compounds of the present disclosure may be modified by, for example, the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection, than do the corresponding unmodified compounds. Such modifications may also increase the compounds' solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently, or in lower doses than with the unmodified compound.

An active agent and a therapeutic agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the disclosure includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The compounds of the present disclosure can optionally be employed in combination with one or more active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents including but not limited to PARP inhibitors, ACAT1 inhibiting compounds, autophagy inhibiting compounds, tyrosine kinase and signaling kinase inhibitors (such as AKT, MEK), ), cell cycle inhibitors (such as CDK4/6, Wee1, Plk, Aurora kinase), and chromatin modifiers.

Further examples of additional therapeutic agents include, without limitation, enzymes, receptor antagonists or agonists, hormones, growth factors, autogenous bone marrow, antibiotics, antimicrobial agents, RNA and DNA molecules and nucleic acids, and antibodies. Specific examples of useful therapeutic agents the above categories include: anti-neoplastics such as androgen inhibitors, antimetabolites, cytotoxic agents, and immunomodulators.

Additional therapeutic agents as used herein also include anti-cancer agents such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics.

Further examples of alkylating antineoplastic agents include carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; vinca alkaloid natural antineoplastics, such as vinblastine and vincristine, and PD1 inhibitors such as lambrolizumab.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

As used herein, the term “modulate” means that the compounds of Formula (I), described herein either increase or decrease the activity of RNA Pol I.

As used herein, the term “hyperproliferative disease” includes cancer and other diseases such as neoplasias and hyperplasias. Cellular proliferative diseases include, for example, rheumatoid arthritis, inflammatory bowel disease, osteoarthritis, leiomyomas, adenomas, lipomas, hemangiomas, fibromas, vascular occlusion, restenosis, artherosclerosis, a pre-neoplastic lesion, carcinoma in situ, oral hairy leukoplakia, or psoriasis. In accordance with one or more embodiments, the term cancer can include any of acute lymphocytic cancer, acute myeloid leukemia, alveolar rhabdomyosarcoma lung, cancer of the blood and heart (e.g., leukemia, lymphoma, carcinoma), cancer of the head and neck, skin cancer, blood and heart (e.g., leukemia, lymphoma, carcinoma), brain cancer, central nervous system cancer, peripheral nerve sheet tumors, breast cancer, cancer of the anus, anal canal, colorectum or anorectum, cancer of the eye, cancer of the intrahepatic bile duct, pancreatic cancer, cancer of the joints, cancer of the neck, gallbladder, or pleura, cancer of the nose, nasal cavity, or middle ear, cancer of the oral cavity, cancer of the vulva, cancer of the prostate, kidney cancer, chronic lymphocytic leukemia, chronic myeloid cancer, colon cancer, esophageal cancer, cervical cancer, breast cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

As used herein, non-limiting examples of autoimmune diseases or disorders include: Acute Disseminated Encephalomyelitis (ADEM); Acute necrotizing hemorrhagic leukoencephalitis; Addison's disease; Agammaglobulinemia; Alopecia areata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune hepatitis; Autoimmune hyperlipidemia; Autoimmune immunodeficiency; Autoimmune inner ear disease (AIED); Autoimmune myocarditis; Autoimmune pancreatitis; Autoimmune retinopathy; Autoimmune thrombocytopenic purpura (ATP); Autoimmune thyroid disease; Autoimmune urticaria; Axonal & neuronal neuropathies; Balo disease; Behcet's disease; Bullous pemphigoid; Cardiomyopathy; Castleman disease; Celiac disease; Chagas disease; Chronic inflammatory demyelinating polyneuropathy (CIDP); Chronic recurrent multifocal osteomyelitis (CRMO); Churg-Strauss syndrome; Cicatricial pemphigoid/benign mucosa pemphigoid; Crohn's disease; Cogans syndrome; Cold agglutinin disease; Congenital heart block; Coxsackie myocarditis; CREST disease; Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic fasciitis; Erythema nodosum; Experimental allergic encephalomyelitis; Evans syndrome; Fibrosing alveolitis; Giant cell arteritis (temporal arteritis); Glomerulonephritis; Goodpasture's syndrome; Granulomatosis with Polyangiitis (GPA) see Wegener's; Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Idiopathic thrombocytopenic purpura (ITP); IgA nephropalothy; IgG4-related sclerosing disease; Immunoregulatory lipoproteins; Inclusion body myositis; Insulin-dependent diabetes (type 1); Interstitial cystitis; Juvenile arthritis; Juvenile diabetes; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Ocularcicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Peripheral neuropathy; Perivenous encephalomyelitis; Pernicious anemia; POEMS syndrome; Polyarteritis nodosa; Type I, II, & III autoimmune polyglandular syndromes; Polymyalgia rheumatica; Polymyositis; Postmyocardial infarction syndrome; Postpericardiotomy syndrome; Progesterone dermatitis; Primary biliary cirrhosis; Primary sclerosing cholangitis; Psoriasis; Psoriatic arthritis; Idiopathic pulmonary fibrosis; Pyoderma gangrenosum; Pure red cell aplasia; Raynauds phenomenon; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sjogren's syndrome; Sperm & testicular autoimmunity; Stiff person syndrome; Subacute bacterial endocarditis (SBE); Susac's syndrome; Sympathetic ophthalmia; Takayasu's arteritis; Temporal arteritis/Giant cell arteritis; Thrombocytopenic purpura (TTP); Tolosa-Hunt syndrome; Transverse myelitis; Ulcerative colitis; Undifferentiated connective tissue disease (UCTD); Uveitis; Vasculitis; Vesiculobullous dermatosis; Vitiligo; Wegener's granulomatosis (Granulo-matosis with Polyangiitis (GPA)).

As used herein, non-limiting examples of a conditions associated with inflammation or pain include: acid reflux, heartburn, acne, allergies and sensitivities, Alzheimer's disease, asthma, atherosclerosis, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, heart disease, hepatitis, high blood pressure, insulin resistance, interstitial cystitis, joint pain/arthritis/rheumatoid arthritis, metabolic syndrome (syndrome X), myositis, nephritis, obesity, osteopenia, osteoporosis, Parkinson's disease, periodontal disease, polyarteritis, polychondritis, psoriasis, scleroderma, sinusitis, Sjogren's syndrome, spastic colon, systemic candidiasis, tendonitis, urinary tract infections, vaginitis, inflammatory cancer (e.g., inflammatory breast cancer) and the like.

### EXAMPLES

Exemplary compounds were prepared via several general synthetic routes set forth in the examples below. Any of the disclosed compounds of the present invention can be prepared according to one or more of these synthetic routes or specific examples, or via modifications thereof accessible to the person of ordinary skill in the art.

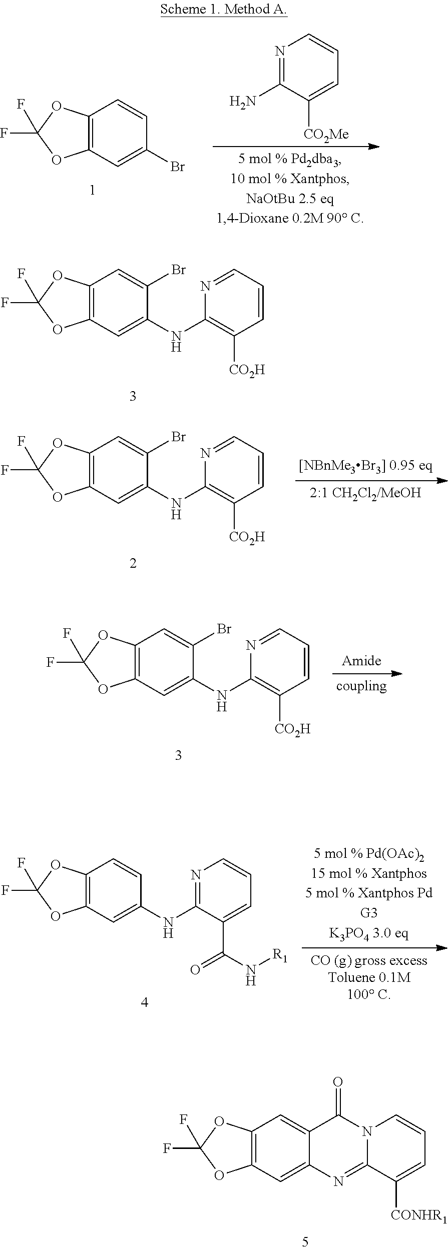

### Intermediate 3:2-((6-bromo-2,2-difluorobenzo[d][1,3]dioxol-5-yl)amino)nicotinic acid

### Step 1: Preparation of 2-((2,2-difluorobenzo[d][1,3]dioxol-5-yl)amino)nicotinic acid

To a 20 mL microwave vial charged with a magnetic stir bar, 5-bromo-2,2-difluoro-1,3-benzodioxole (500 mg, 2.1 mmol), methyl 2-aminopyridine-3-carboxylate (320 mg, 2.1 mmol), Tris(dibenzylideneacetone)dipalladium(O) (97 mg, 0.10 mmol), and Xantphos (120 mg, 0.21 mmol) was added 1,4-Dioxane (11 mL) [deoxygenated by bubbling nitrogen for 5 min prior to initiation]. To this reaction mixture was added Sodium t-butoxide (510 mg, 5.3 mmol). Sealed reaction with teflon cap. Heated to 90° C. for 2 h. The reaction was monitored by LCMS. Upon completion, desired mass of hydrolized product [M+1=295] is observed. Poured into brine, extracted with ethyl acetate, dried over sodium sulfate, filtered off solids, concentrated under reduced pressure. Purified by automated normal phase chromatography (0-100% ethyl acetate/heptane). 350 mg, 56% yield. MS, ES⁺ m/z=295.0, ¹H NMR (400 MHz, DMSO-d6) δ ppm 6.91 (dd, J=7.71, 4.80 Hz, 1H) 7.28-7.37 (m, 2H) 8.04 (d, J=1.96 Hz, 1H) 8.26 (dd, J=7.74, 1.99 Hz, 1H) 8.39 (dd, J=4.77, 1.99 Hz, 1H) 10.47 (s, 1H).

### Step 2: Preparation of 2-((6-bromo-2,2-difluorobenzo[d][1,3]dioxol-5-yl)amino)nicotinic acid

Benzyltrimethylammonium tribromide (230 mg, 0.59 mmol, 0.5 equivalents) was added to a solution of 2-((2,2-difluorobenzo[d][1,3]dioxol-5-yl)amino)nicotinic acid (350 mg, 1.2 mmol) in 2:1 dichloromethane (8 mL)/methanol (4 mL) at room temperature. Stirred for 5 minutes. The reaction was monitored by LCMS. The expected mass for mono-brominated product is observed [M+1]=373, unreacted starting material remains. Added another 0.45 equivalents for a total of 0.95 equivalents. Upon completion, the crude reaction was poured into water, acidified to pH 1 using 1N HCl aq., extracted with ethyl acetate, dried over sodium sulfate, filtered off solids, and concentrated under reduced pressure. Purified by automated reverse phase chromatography (5-95% acetonitrile/water; 0.05% TFA buffer). The product-containing fractions were combined. Concentrated under reduced pressure to give the title compound 320 mg, 72% yield. MS, ES⁺ m/z=373.0, ¹H NMR (400 MHz, DMSO-d6) δ ppm 6.99 (dd, J=7.74, 4.77 Hz, 1H) 7.89 (s, 1H) 8.31 (dd, J=7.77, 1.96 Hz, 1H) 8.42 (dd, J=4.83, 2.05 Hz, 1H) 8.58 (s, 1H) 10.68 (s, 1H).

### Example 1: 2,2-difluoro-11-oxo-N-(2-(pyrrolidin-1-yl)ethyl)-11H-[1,3]dioxolo[4,5-2]pyrido[2,1-b]quinazoline-6-carboxamide; hydrochloride (Compound 107)

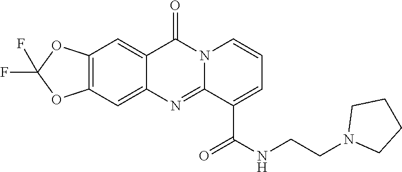 [SMILES: O=C(NCCN1CCCC1)c1cccn2c(=O)c3cc4c(cc3nc12)OC(F)(F)O4]

### Method A

### Step 1: Preparation of 2-((6-bromo-2,2-difluorobenzo[d][1,3]dioxol-5-yl)amino)-N-(2-(pyrrolidin-1-yl)ethyl)nicotinamide

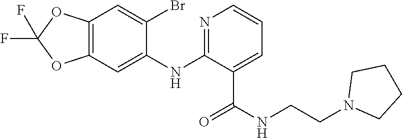 [SMILES: O=C(NCCN1CCCC1)c1cccnc1Nc1cc2c(cc1Br)OC(F)(F)O2]

To a 20 mL scintillated vial charged with a magnetic stir bar, 2-((6-bromo-2,2-difluorobenzo[d][1,3]dioxol-5-yl) amino)nicotinic acid (50 mg, 0.13 mmol) was taken up in dimethylformamide (1.0 mL). To this solution was added N-ethyl-N-isopropylpropan-2-amine (72 uL, 0.40 mmol), followed by 2-(1H-Benzotriazole-1-yl)-1,1,3,3-tetramethylaminium tetrafluoroborate TBTU (65 mg, 0.20 mmol) and 2-pyrrolidin-1-ylethanamine (28 uL, 0.20 mmol). The reaction was stirred at room temp for 24 h and monitored by LCMS. Upon completion, the crude reaction was diluted with ethyl acetate, poured into water. The organic phase was washed with water, followed by brine, dried over sodium sulfate, filtered off solids, and concentrated under reduced pressure. Purified by automated reverse phase chromatography (5-95% acetonitrile/water; 0.05% TFA buffer). The product-containing fractions were combined. Concentrated under reduced pressure. MS, ES+ m/z=469.1

### Step 2: Preparation of 2,2-difluoro-11-oxo-N-(2-(pyrrolidin-1-yl)ethyl)-11H-[1,3]dioxolo[4,5-g]pyrido[2,1-b]quinazoline-6-carboxamide hydrochloride

To a 20 mL microwave vial with a septum were added of 2-[(6-bromo-2,2-difluoro-1,3-benzodioxol-5-yl)amino]-N-(2-pyrrolidin-1-ylethyl)pyridine-3-carboxamide (135 mg, 0.29 mmol) and degassed toluene (6 mL). Pd(OAc)₂ (3.2 mg, 0.01 mmol), XantPhos (25 mg, 0.04 mmol), XantPhos Pd G3 (13.6 mg, 0.01 mmol) and K₃PO₄ (177 mg, 0.83 mmol) were added while the solution was degassed with N₂. The septum was replaced by the seal, and the mixture was degassed with CO for 5 min. The mixture was heated at 100° C. for 18 h, then cooled to rt. The mixture was concentrated under reduced pressure. The material was purified by prep-HPLC using MeCN and 10 mM AmBic pH 10. The product-containing fractions were combined. The material was taken in DCM (5 mL) and HCl (4 N in dioxane, 0.15 mL, 0.58 mmol) was added. The mixture was concentrated under reduced pressure and lyophilized to provide title compound (28 mg, 22%) as a yellow solid. ¹H NMR (400 MHz, CD₃OD) δ 9.14 (dd, J=7.3, 1.7 Hz, 1H), 8.78 (dd, J=7.1, 1.7 Hz, 1H), 8.11 (s, 1H), 7.86 (s, 1H), 7.24 (t, J=7.2 Hz, 1H), 3.97 (t, J=5.9 Hz, 2H), 3.88-3.81 (m, 2H), 3.57 (t, J=5.9 Hz, 2H), 3.25-3.17 (m, 2H), 2.26-2.15 (m, 2H), 2.10-2.00 (m, 2H). MS, ES+ m/z=417.2

The following compounds were synthesized according to Method A

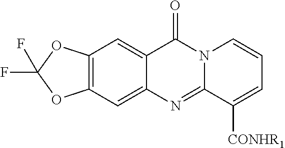

| | Compd | | | Data | Preparation |
|---|---|---|---|---|---|
| Ex | No. | Name | R₁ | Analytical | Information |
| 2 | 106 | N-(2-(dimethylamino)- 2-methylpropyl)-2,2- difluoro-11-oxo-11H- [1,3]dioxolo[4,5-g] | 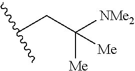 [SMILES: *CC(C)(C)N(C)C] | MS, ES⁺ m/z = 419.2, ¹H NMR (400 MHz, CD₃OD) δ 9.09 (dd, J = 7.2, 1.6 Hz, 1H), 8.74 (dd, J = 7.1, 1.6 Hz, 1H), | Method A using N²,N²,2-trimethylpropane- 1,2-diamine (56.1 mg, 0.482 mmol) and intermediate 3 |
| | | pyrido[2,1-b] | | 8.06 (s, H), 7.64 (s, 1H), | (150 mg, 0.402 mmol) |
| | | quinazoline-6- | | 7.23 (t, J = 7.2, Hz, 1H), | to give 95 mg, 50% yield. |
| | | carboxamide; | | 3.97 (s, 2H), 2.98 (s, 6H), | |
| | | hydrochloride | | 1.57 (s, 6H). | |
| 3 | 105 | 2,2-difluoro-N-(2-(4- hydroxypiperidin-1-yl) ethyl)-11-oxo-11H- [1,3]dioxolo[4,5-g] pyrido[2,1-b] quinazoline-6- | 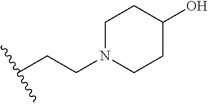 [SMILES: *CCN1CCC(O)CC1] | MS, ES⁺ m/z = 447.1, ¹H NMR (400 MHz, CD₃OD) δ 9.21 (d, J = 7.2 Hz, 1H), 8.86 (d, J = 6.9 Hz, 1H), 8.14 (s, 1H), 7.92 (s, 1H),7 .41-7.34 (m, 1H), 4.02-3.95 (m, 2H), 3.89-3.73 (m, 2H), | Method A using 1-(2-aminoethyl)piperidin-4-ol; dihydrochloride (489 mg, 2.25 mmol) and intermediate 3 (560 mg, 1.50 mmol) to give 640 mg, 85% yield. |
| | | carboxamide; | | 3.69-3.62 (m, 1H), 3.52-3.46 (m, 2H), | |
| | | hydrochloride | | 3.44-3.36 (m, 1H), 3.19-3.12 (m, 1H), | |
| | | | | 2.22-2.15 (m, 1H), 2.13-2.04 (m, 1H), | |
| | | | | 2.00-1.93 (m, 1H), 1.85-1.75 (m, 1H). | |
| 4 | 102 | 2,2-difluoro-N-(2-(3- hydroxypyrrolidin- 1-yl)ethyl)-11-oxo- 11H-[1,3]dioxolo [4,5-g]pyrido[2,1-b] quinazoline-6- carboxamide; | 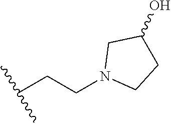 [SMILES: *CCN1CCC(O)C1] | MS, ES⁺ m/z = 433.1, ¹H NMR (400 MHz, CD₃OD) δ 9.36-9.25 (m, 1H), 9.01-8.88 (m, 1H), 8.27-8.10 (m, 1H), 8.00-7.94 (m, 1H), 7.62-7.47 (m, 1H), 4.65-4.55 (m, 1H), 4.01-3.88 (3.5H), 3.85-3.80 (m, 0.5), 3.68-3.54 (m, 2H), 3.52-3.42 | Method A using 1-(2-aminoethyl)pyrrolidin-3-ol (398 mg, 3.06 mmol) and intermediate 3 (600 mg, 3.06 mmol) to give 155 mg, 58% yield. |
| | | hydrochloride | | (m, 0.5H), 3.40-3.33 (m, 0.5H), | |
| | | | | 3.29-3.21 (m, 1H), 2.47-2.37 | |
| | | | | (m, 0.5H), 2.22-2.03 (m, 1.5H). | |
| 5 | 104 | N-(2-(dimethylamino) ethyl)-2,2-difluoro- 11-oxo-11H- [1,3]dioxolo[4,5-g] | 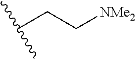 [SMILES: *CCN(C)C] | MS, ES⁺ m/z = 391.1, ¹H NMR (400 MHz, CD₃OD) δ 9.13 (dd, J =7.3, 1.7 Hz, 1H), 8.78 (dd, J = 7.1, 1.7 Hz, 1H), | Modification of Method A using HATU, N¹, N¹- dimethylethane-1,2-diamine (33 μl, 0.302 mmol) |
| | | pyrido[2,1-b] | | 8.11 (s, 1H), 7.86 (s, 1H), | and intermediate 3 |
| | | quinazoline-6- | | 7.24 (t, J = 7.2 Hz, 1H), | (75 mg, 0.201 mmol) |
| | | carboxamide; | | 4.01-3.95 (m, 2H), 3.54-3.48 | to give 92 mg, 99% yield. |
| | | hydrochloride | | (m, 2H), 3.04 (s, 6H). | |

### Example 6: 2,2-difluoro-11-oxo-N-(2-(4-oxopiperidin-1-yl)ethyl)-11H-[1,3]dioxolo[4,5-g]pyrido[2,1-b]quinazoline-6-carboxamide (Compound 103)

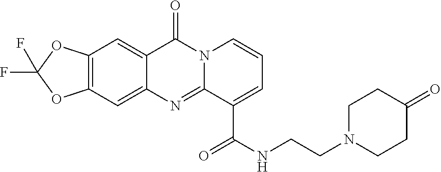 [SMILES: O=C1CCN(CCNC(=O)c2cccn3c(=O)c4cc5c(cc4nc23)OC(F)(F)O5)CC1]

To a mixture of 14,14-difluoro-N-[2-(4-hydroxy-1-piperidyl)ethyl]-9-oxo-13,15-dioxa-2,8-diazatetracyclo[8.7.0.03,8.012,16]heptadeca-1(17),2,4,6,10,12(16)-hexaene-4-carboxamide;hydrochloride compound 105 (117 mg, 0.242 mmol) in DCM (1.5 mL) and DMSO (3.0 mL) was added pyridine sulfur trioxide complex (386 mg, 2.42 mmol) at room temperature. The mixture heated to 60° C., stirred for 3 h, and then cooled to room temperature. The volatiles were removed under reduced pressure. The residue was purified on silica gel (C18) using MeCN and 10 mM AmBic pH 10. The product containing fractions were lyophilized. The material was taken in DCM and HCl (4 N in dioxane, 0.24 mL, 0.968 mmol) was added. The mixture was concentrated under reduced pressure and lyophilized to provide title compound (64 mg, 55%) as a solid. ¹H NMR (400 MHz, CD₃OD) δ 9.21 (d, J=7.1 Hz, 1H), 8.86 (d, J=7.1 Hz, 1H), 8.14 (s, 1H), 7.92 (s, 1H), 7.38 (t, J=7.1 Hz, 1H), 3.99 (dd, J=9.3, 5.8 Hz, 2H), 3.8-3.68 (m, 2H), 3.57-3.47 (m, 2H), 3.28-3.14 (m, 2H), 2.23-2.10 (m 2H), 2.06-1.96 (m, 2H). MS, ES⁺ m/z=445.2

### Example 7: N-(2-((3R,4R)-3,4-dihydroxypyrrolidin-1-yl)ethyl)-2,2-difluoro-11-oxo-11H-[1,3]dioxolo[4,5-g]pyrido[2,1-b]quinazoline-6-carboxamide (Compound 101)

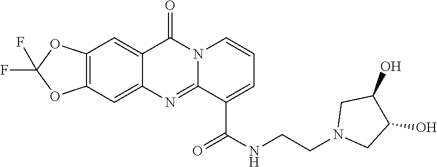 [SMILES: O=C(NCCN1C[C@@H](O)[C@H](O)C1)c1cccn2c(=O)c3cc4c(cc3nc12)OC(F)(F)O4]

### Step 1: Preparation of 2-((6-bromo-2,2-difluorobenzo[d][1,3]dioxol-5-yl)amino)-N-(2-hydroxyethyl)nicotinamide

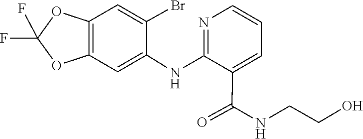 [SMILES: O=C(NCCO)c1cccnc1Nc1cc2c(cc1Br)OC(F)(F)O2]

To a solution of 2-[(6-bromo-2,2-difluoro-1,3-benzodioxol-5-yl)amino]pyridine-3-carboxylic acid (300 mg, 0.804 mmol) in DMF (6.00 mL) were added N-ethyl-N-isopropylpropan-2-amine (0.413 mL, 2.41 mmol), followed by 1-[Bis(dimethylamino)methylene]-1H-1,2,3-triazolo[4,5-b]pyridinium 3-oxid hexafluorophosphate HATU (459 mg, 1.21 mmol) and 2-aminoethanol (0.07 mL, 1.21 mmol). The reaction was stirred at rt for 1 h, then water (20 mL) was added. The suspension was filtered. The solid was rinsed with water (15 m) and dried under reduced pressure to provide title compound (204 mg, 61%) as a solid. ¹H NMR (500 MHz, DMSO-d₆) δ 11.14 (s, 1H), 8.79 (s, 1H), 8.54 (s, 1H), 8.33 (d, J=3.2 Hz, 1H), 8.18 (d, J=6.5 Hz, 1H), 7.83 (s, 1H), 6.98 (dd, J=7.6, 4.8 Hz, 1H), 4.76 (s, 1H), 3.54 (s, 2H), 3.39-3.34 (m, 2H). MS, ES+ m/z=416.1.

### Step 2: Preparation of 2-((6-bromo-2,2-difluorobenzo[d][1,3]dioxol-5-yl)amino)-N-(2-oxoethyl)nicotinamide

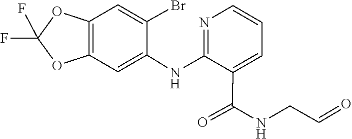 [SMILES: O=CCNC(=O)c1cccnc1Nc1cc2c(cc1Br)OC(F)(F)O2]

To a suspension of 2-[(6-bromo-2,2-difluoro-1,3-benzodioxol-5-yl)amino]-N-(2-hydroxyethyl)pyridine-3-carboxamide (200 mg, 0.481 mmol) in DCM (8.00 mL) was added Dess-Martin Periodinane (306 mg, 0.721 mmol) at room temperature. The mixture was stirred for 3 h, then a saturated aqueous solution of NaHCO₃ (10 mL) and Na₂S₂O₃ (10 mL) were added. The separated aqueous layer was extracted with DCM. The combined organic layers were dried (MgSO₄), filtered and concentrated under reduced pressure to provide title compound (200 mg) and was used without further purification for the next step. MS, ES+ m/z=414.1

### Step 3: 2-((6-bromo-2,2-difluorobenzo[d][1,3]dioxol-5-yl)amino)-N-(2-((3R,4R)-3,4-dihydroxypyrrolidin-1-yl)ethyl)nicotinamide

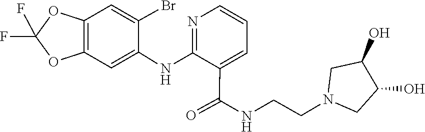 [SMILES: O=C(NCCN1C[C@@H](O)[C@H](O)C1)c1cccnc1Nc1cc2c(cc1Br)OC(F)(F)O2]

To the crude material in MeOH (5.00 mL) was added (3 S,4S)-pyrrolidine-3,4-diol (59.5 mg, 0.58 mmol) at 0° C. After 30 min, NaBH₃CN (30.2 mg, 0.481 mmol) was added at 0° C. The reaction mixture was slowly warmed to rt and stirred for 18 h. The reaction was concentrated under reduced pressure. The material was purified on silica gel (C18) using MeCN and 0.1% HCOOH in water to provide title compound (40 mg, 16%) as a solid. MS, ES+ m/z=501.1

### Step 4: Preparation of N-(2-((3R,4R)-3,4-dihydroxypyrrolidin-1-yl)ethyl)-2,2-difluoro-11-oxo-11H-[1,3]dioxolo[4,5-g]pyrido[2,1-b]quinazoline-6-carboxamide

The titled compound was prepared according to Method A step 2 using 2-((6-bromo-2,2-difluorobenzo[d][1,3]dioxol-5-yl)amino)-N-(2-((3R,4R)-3,4-dihydroxypyrrolidin-1-yl)ethyl)nicotinamide to give 4 mg, 57% yield. MS, ES⁺ m/z=449.1, ¹H NMR (500 MHz, CD₃OD) δ 9.06 (dd, J=7.2, 1.6 Hz, 1H), 8.70 (dd, J=7.0, 1.6 Hz, 1H), 8.03 (s, 1H), 7.86 (s, 1H), 7.19 (t, J=7.1 Hz, 1H), 4.16-4.12 (m, 2H), 3.75-3.62 (m, 2H), 3.16 (dd, J=10.2, 5.3 Hz, 2H), 2.94-2.85 (m, 2H), 2.73 (dd, J=10.5, 3.0 Hz, 2H).

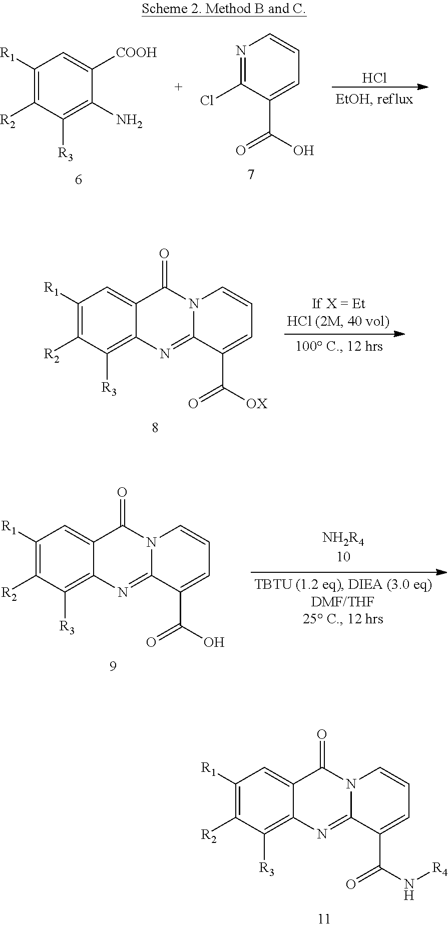

### Example 8: 12-oxo-N-(2-(pyrrolidin-1-yl)ethyl)-2,3-dihydro-12H-[1,4]dioxino[2,3-2]pyrido[2,1-b]quinazoline-7-carboxamide; hydrochloride (Compound 113)

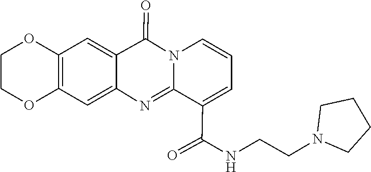 [SMILES: O=C(NCCN1CCCC1)c1cccn2c(=O)c3cc4c(cc3nc12)OCCO4]

### Method B

### Step 1: Preparation of ethyl 12-oxo-2,3-dihydro-12H-[1,4]dioxino[2,3-g]pyrido[2,1-b]quinazoline-7-carboxylate

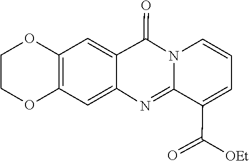 [SMILES: CCOC(=O)c1cccn2c(=O)c3cc4c(cc3nc12)OCCO4]

To a solution of 7-amino-2,3-dihydrobenzo[b][1,4]dioxine-6-carboxylic acid (1.00 g, 5.12 mmol, 1.00 eq) and 2-chloronicotinic acid (807 mg, 5.12 mmol, 1.00 eq) in ethanol (20 mL) was added hydrochloric acid (37%, 505 mg, 5.12 mmol, 1.00 eq). The mixture was stirred at 90° C. for 12 hrs. The reaction was monitored by LCMS. Upon completion the reaction mixture was concentrated under reduced pressure to give a residue and was purified by prep-HPLC (TFA condition; column: Phenomenex luna C18 250*50 mm*10 um; mobile phase: [water (0.1% TFA)-acetonitrile]; B %: 10%-35%, 18 min.) to give ethyl 12-oxo-2,3-dihydro-12H-[1,4]dioxino[2,3-g]pyrido[2,1-b]quinazoline-7-carboxylate (1.25 g, 3.83 mmol, 74.8% yield) as yellow oil. MS, ES⁺ m/z=327.1.

### Step 2: 12-oxo-2,3-dihydro-12H-[1,4]dioxino[2,3-g]pyrido[2,1-b]quinazoline-7-carboxylic acid

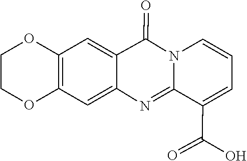 [SMILES: O=C(O)c1cccn2c(=O)c3cc4c(cc3nc12)OCCO4]

Ethyl 12-oxo-2,3-dihydro-12H-[1,4]dioxino[2,3-g]pyrido[2,1-b]quinazoline-7-carboxylate (0.50 g, 1.53 mmol, 1.00 eq) was added HCl aq. (2 M, 20 mL, 26.10 eq) in H₂O (20 mL). The mixture was stirred at 100° C. for 12 hrs. The reaction was monitored by LCMS. Starting material was consumed, and one major new peak with desired MS was detected. Upon completion, the reaction mixture was concentrated under reduced pressure to give 12-oxo-2,3-dihydro-12H-[1,4]dioxino[2,3-g]pyrido[2,1-b]quinazoline-7-carboxylic acid (0.31 g, crude) as a yellow solid. MS, ES⁺ m/z=299.0.

### Step 3: 12-oxo-N-(2-(pyrrolidin-1-yl)ethyl)-2,3-dihydro-12H-[1,4]dioxino[2,3-g]pyrido[2,1-b]quinazoline-7-carboxamide

To a solution of 12-oxo-2,3-dihydro-12H-[1,4]dioxino[2,3-g]pyrido[2,1-b]quinazoline-7-carboxylic acid (70.0 mg, 235 umol, 1 eq) in dimethylformamide (3.0 mL) was added TBTU (90 mg, 280 umol, 1.20 eq) and DIEA (91.0 mg, 700 umol, 3.00 eq). The mixture was added to compound 5 (40 mg, 350 umol, 1.50 eq) in THE (1.0 mL). The mixture was stirred at 25° C. for 12 hrs. The reaction was monitored by LCMS Upon completion, the reaction mixture was concentrated under reduced pressure, and purified by prep-HPLC (HCl condition; column: Luna C18 100*30 5 u; mobile phase: [water (0.04% HCl)-acetonitrile]; B %: 1%-30%, 15 min). (62.0 mg, 157 umol, 67.0% yield) was obtained as a yellow solid. MS, ES⁺ m/z=395.1, ¹H NMR (400 MHz, MeOD) δ 9.38 (d, J=6.4 Hz, 1H), 9.03 (d, J=7.2 Hz, 1H), 7.89 (s, 1H), 7.67 (t, J=7.2 Hz, 1H), 7.49 (s, 1H), 4.55-4.45 (m, 4H), 3.95-3.90 (m, 4H), 3.59 (t, J=5.6 Hz, 2H), 3.25-3.21 (m, 2H), 2.23-2.09 (m, 4H).

### Example 9: 3-methyl-11-oxo-N-(2-(pyrrolidin-1-yl)ethyl)-11H-pyrido[2,1-b]quinazoline-6-carboxamide;hydrochloride (Compound 116)

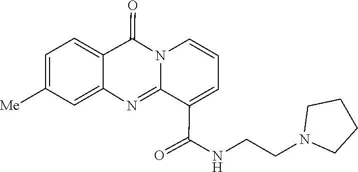 [SMILES: Cc1ccc2c(=O)n3cccc(C(=O)NCCN4CCCC4)c3nc2c1]

### Method C

### Step 1: Preparation of 3-methyl-11-oxo-pyrido[2,1-b]quinazoline-6-carboxylic acid

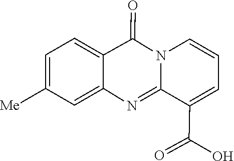 [SMILES: Cc1ccc2c(=O)n3cccc(C(=O)O)c3nc2c1]

In a vial, at room temperature, to a suspension of 2-chloropyridine-3-carboxylic acid (98%, 110 mg, 0.684 mmol) and 2-amino-4-methylbenzoic acid (97%, 106 mg, 0.680 mmol) in Ethanol (3 mL), was added dropwise 12 M hydrogen chloride 37% (150 uL, 1.80 mmol). The reaction mixture was stirred at 80° C. for 2 days, then cooled at room temperature. 12 M hydrogen chloride 37% (400 uL, 4.80 mmol) and EtOH (0.5 mL) were added. The yellow suspension was stirred 1.5 day more at 80° C. The yellow suspension was cooled down and filtered. The yellow precipitate was washed with EtOH and dried in vacuum to give 3-methyl-11-oxo-pyrido[2,1-b]quinazoline-6-carboxylic acid (85 mg, 43% Yield) as a yellow solid. MS, ES⁺ m/z=255.0, ¹H NMR (400 MHz, DMSO-d6) δ 9.07 (dd, J=7.2, 1.6 Hz, 1H), 8.66 (dd, J=7.0, 1.6 Hz, 1H), 8.25 (d, J=8.3 Hz, 1H), 7.73 (s, 1H), 7.47 (dd, J=8.3, 1.3 Hz, 1H), 7.29 (t, J=7.1 Hz, 1H), 2.55 (s, 3H).

### Step 2: Preparation of 3-methyl-11-oxo-N-(2-pyrrolidin-1-ylethyl)pyrido[2,1-b]quinazoline-6-carboxamide

In a vial, under inert atmosphere, to a suspension of 3-methyl-11-oxo-pyrido[2,1-b]quinazoline-6-carboxylic acid;hydrochloride (58 mg, 0.200 mmol) in DMF-Anhydrous (2 mL) was added [benzotriazol-1-yloxy(dimethylamino)methylene]-dimethyl-ammonium;tetrafluoroborate (98%, 152 mg, 0.464 mmol) and N-ethyl-N-isopropyl-propan-2-amine (100 uL, 0.573 mmol). The reaction mixture was stirred 5 min at room temperature and N-(2-aminoethyl)pyrrolidine (98%, 31 uL, 0.239 mmol) was added. The reaction mixture was stirred overnight at room temperature. The yellow precipitate formed was filtered, washed with Et2O and dried in vacuum to give 3-methyl-11-oxo-N-(2-pyrrolidin-1-ylethyl)pyrido[2,1-b]quinazoline-6-carboxamide;hydrochloride (18 mg, 23% Yield) as a yellow powder. MS, ES⁺ m/z=351.0, ¹H NMR (400 MHz, DMSO-d6) δ 11.14 (s, 1H), 10.20 (s, 1H), 9.02 (dd, J=7.2, 1.7 Hz, 1H), 8.62 (dd, J=7.0, 1.7 Hz, 1H), 8.24 (d, J=8.3 Hz, 1H), 7.89 (s, 1H), 7.44 (dd, J=8.3, 1.4 Hz, 1H), 7.20 (t, J=7.1 Hz, 1H), 3.86 (d, J=6.0 Hz, 2H), 3.66 (s, 2H), 3.52-3.38 (m, 2H), 3.10 (s, 2H), 2.56 (s, 3H), 1.95 (dd, J=40.5, 7.6 Hz, 4H).

### Step 3: Preparation of 3-methyl-11-oxo-N-(2-pyrrolidin-1-ylethyl)pyrido[2,1-b]quinazoline-6-carboxamide;hydrochloride

In a round bottom flask, under inert atmosphere, 3-methyl-11-oxo-N-(2-pyrrolidin-1-ylethyl)pyrido[2,1-b]quinazoline-6-carboxamide;hydrochloride (18 mg, 0.0465 mmol) was solubilized in DCM (0.9057 mL) and Methanol (0.9057 mL) and 2 M hydrogen chloride in Et2O 2N (91 uL, 0.181 mmol) was added dropwise in the reaction mixture which was stirred 1 h30 at room temperature. Diethyl ether was added, and the precipitate was filtered, washed with diethyl ether and dried in vacuum to give 3-methyl-11-oxo-N-(2-pyrrolidin-1-ylethyl)pyrido[2,1-b]quinazoline-6-carboxamide;hydrochloride (18.6 mg, 10000 Yield) as a yellow solid. MS, ES⁺ m/z=351.0, 3H NVR (DMSO-d6, 500 M MHz): δ (ppm) 10.91-11.24 (m, 1H), 10.08-10.63 (m, 1H), 9.02 (dd, J=7.2, 1.6 Hz, 1H), 8.62 (dd, J=7.1, 1.7 Hz, 1H), 8.23 (d, J=8.1 Hz, 1H), 7.91 (s, 1H), 7.44 (dd, J=8.3, 1.5 Hz, 1H), 7.20 (t, J=7.1 Hz, 1H), 3.86 (q, J=6.2 Hz, 2H), 3.58-3.73 (m, 2H), 3.45 (q, J=6.0 Hz, 2H), 3.02-3.16 (m, 2H), 2.56 (s, 3H), 1.96-2.07 (m, 2H), 1.82-1.93 (m, 2H).

The following compounds were synthesized according to Method B or Method C.

| | Com- | | | |
|---|---|---|---|---|
| | pound | | | Preparation |
| Ex | No. | Name | Analytical Data | Information |
| 10 | 114 | N-(2- | MS, ES⁺ m/z = 369.1, ¹H NMR | Method B using 7- |
| | | (dimethylamino)ethyl)- | (400 MHz, MeOD) δ 9.34 (d, J = | amino-2,3- |
| | | 12-oxo-2,3- | 7.2 Hz, 1H), 8.92 (d, J = 7.2 Hz, | dihydrobenzo[b][1,4] |
| | | dihydro-12H- | 1H), 7.90 (s, 1H), 7.57 (t, J = 7.2 | dioxine-6-carboxylic |
| | | [1,4]dioxino[2,3- | Hz), 7.49 (s, 1H), 4.53-4.43 (m, | acid, 2- |
| | | g]pyrido[2,1- | 4H), 3.95 (t, J = 6.0 Hz, 2H), 3.53 | chloronicotinic acid, |
| | | b]quinazoline-7- | (t, J = 6.0 Hz, 2H), 3.05 (s, 6H). | N¹,N¹- |
| | | carboxamide; hydro- | | dimethylethane-1,2- |
| | | chloride | | diamine to give 28 |
| | | | | mg. |
| 11 | 108 | 11-oxo-N-(2- | MS, ES⁺ m/z = 381.1, ¹H NMR | Method B using 6- |
| | | (pyrrolidin-1- | (400 MHz, DMSO-d6) δ 11.31 | aminobenzo[d][1,3]dioxole- |
| | | yl)ethyl)-11H- | (s, 1H), 8.96 (m, 1H), 8.58 (m, | 5-carboxylic |
| | | [1,3]dioxolo[4,5- | 1H), 7.70-7.56 (m, 2H) 7.20 (s, | acid, 2- |
| | | g]pyrido[2,1- | 1H), 6.28 (s, 2H), 3.54 (s, 2H), | chloronicotinic acid, |
| | | b]quinazoline-6- | 2.70-2.50 (m, 6H), 1.81 (b. s, | and 2-(pyrrolidin-1- |
| | | carboxamide; hydro- | 4H). | yl)ethan-1-amine to |
| | | chloride | | give 1.3 mg. |
| 12 | 109 | N-(2- | MS, ES⁺ m/z = 339.1, ¹H NMR | Method B using 2- |
| | | (dimethylamino)ethyl)- | (400 MHz, DMSO-d6) δ 11.3 (s, | amino-4,5- |
| | | 2,3-dimethyl- | 1H), 8.90 (d, J = 7.2, 1H) 8.53 (d, | dimethylbenzoic acid, |
| | | 11-oxo-11H- | J = 6.4 Hz, 1H), 7.95 (s, 1H) 7.51 | 2-chloronicotinic |
| | | pyrido[2,1- | (s, 1H), 7.11 (t, J = 7.2 Hz, 1H), | acid, and N¹,N¹- |
| | | b]quinazoline-6- | 3.55 (m, 2H), 2.68 (m, 2H), 2.53 | dimethylethane-1,2- |
| | | carboxamide; hydro- | (m, 12H). | diamine to give 23.3 |
| | | chloride | | mg. |
| 13 | 110 | 2,3-dimethyl-11- | MS, ES⁺ m/z = 365.2, ¹H NMR | Method B using 2- |
| | | oxo-N-(2- | (400 MHz, MeOD) δ 8.95 (d, J = | amino-4,5- |
| | | (pyrrolidin-1- | 6.8 Hz, 1H), 8.58 (d, J = 6.8 Hz, | dimethylbenzoic acid, |
| | | yl)ethyl)-11H- | 1H), 8.28 (b. s, 1H) 7.97 (s, 1H), | 2-chloronicotinic |
| | | pyrido[2,1- | 7.55 (s, 1H), 7.06 (t, J = 6.8 Hz, | acid, and 2- |
| | | b]quinazoline-6- | 1H), 3.95 (m, 2H) 3.94-3.52 (m, | (pyrrolidin-1- |
| | | carboxamide; hydro- | 6H), 2.42 (d, 6H) 2.121 (s, 4H). | yl)ethan-1-amine to |
| | | chloride | | give 23.9 mg. |
| 14 | 111 | N-(2- | MS, ES⁺ m/z = 365.1, ¹H NMR | Method B using 3- |
| | | (dimethylamino)ethyl)- | (400 MHz, DMSO-d6) δ 10.98 | amino-5,6,7,8- |
| | | 12-oxo- | (m, 1H), 10.78 (b. s, 1H), 8.99 | tetrahydronaphthalene- |
| | | 7,9,10,12- | (dd, J = 7.2, 1.2 Hz, 1H), 8.61 (d, | 2-carboxylic acid, |
| | | tetrahydro-8H- | J = 5.6 Hz, 1H), 8.00 (s, 1H) 7.80 | 2-chloronicotinic |
| | | benzo[g]pyrido[2,1- | (s, 1H), 7.20 (t, J = 7.2 Hz, 1H), | acid, and N¹,N¹- |
| | | b]quinazoline-4- | 3.87 (q, J = 6.0 Hz, 2H), 3.37 (q, | dimethylethane-1,2- |
| | | carboxamide; hydro- | J = 5.6 Hz, 2H), 2.97-2.94 (m, | diamine to give 39 |
| | | chloride | 4H), 2.85 (d, 6H), 1.815 (s, 4H). | mg. |
| 15 | 112 | 12-oxo-N-(2- | MS, ES⁺ m/z = 391.1, ¹H NMR | Method B using |
| | | (pyrrolidin-1- | (400 MHz, MeOD) δ 9.30 (d, J = | amino-5,6,7,8- |
| | | yl)ethyl)-7,9,10,12- | 6.4 Hz, 1H), 8.90 (d, J = 7.2 Hz, | tetrahydronaphthalene- |
| | | tetrahydro-8H- | 1H), 8.20 (s, 1H), 7.70 (s, 1H), | 2-carboxylic acid, |
| | | benzo[g]pyrido[2,1- | 7.48 (b. s, 1H), 3.97-3.88 (m, | 2-chloronicotinic |
| | | b]quinazoline-4- | 4H), 3.58 (t, J = 6 Hz, 2H), 3.24 | acid, and 2- |
| | | carboxamide; hydro- | (b. s, 2H) 3.08-3.04 (m, 4H), | (pyrrolidin-1- |
| | | chloride | 2.22-2.11 (m, 4H), 1.94-1.93 (m, | yl)ethan-1-amine to |
| | | | 4H). | give 14.6 mg. |
| 16 | 115 | (R)-N-(2-(3- | MS, ES⁺ m/z = 381.0, 1H NMR | Method C (with |
| | | hydroxypyrrolidin- | (DMSO-d6, 600 MHz): δ (ppm) | modified step 1 as |
| | | 1-yl)ethyl)-3,4- | 11.13 (br s, 1H), 10.10-10.72 (m, | follows: 1M HCl as |
| | | dimethyl-11-oxo- | 1H), 9.00 (dd, J = 7.2, 1.8 Hz, | solvent, heating to |
| | | 11H-pyrido[2,1- | 1H), 8.59 (dd, J = 7.0, 1.6 Hz, | 110° C.) |
| | | b]quinazoline-6- | 1H), 8.10 (d, J = 8.1 Hz, 1H), | using 2-amino-3,4- |
| | | carboxamide; hydro- | 7.43 (d, J = 8.4 Hz, 1H), 7.20 (t, | dimethylbenzoic acid, |
| | | chloride | J = 7.1 Hz, 1H), 5.47 (br d, J = | 2-chloronicotinic |
| | | | 0.6 Hz, 1H), 4.42 (br s, 1H), | acid, and (R)-1-(2- |
| | | | 3.79-3.95 (m, 2H), 3.36-3.76 (m, | aminoethyl)pyrrolidin- |
| | | | 4H), 2.87-3.25 (m, 2H), 2.57 (s, | 3-ol to give 17 mg, |
| | | | 3H), 2.51 (s, 3H), 1.74-2.33 (m, | 91% Yield as a |
| | | | 2H). | yellow solid. |
| 17 | 120 | 3,4-dimethyl-11- | MS, ES⁺ m/z = 364.0, ¹H NMR | Method C (with |
| | | oxo-N-(2- | (600 MHz, DMSO-d6) δ ppm | modified step 1 as |
| | | (pyrrolidin-1- | 11.14 (t, J = 5.80 Hz, 1 H) 9.88- | follows: 1M HCl as |
| | | yl)ethyl)-11H- | 10.37 (m, 1 H) 9.01 (dd, J = 7.19, | solvent, heating to |
| | | pyrido[2,1- | 1.76 Hz, 1 H) 8.60 (dd, J = 6.97, | 105° C.) using 2- |
| | | b]quinazoline-6- | 1.69 Hz, 1 H) 8.11 (d, J = 8.22 Hz, | amino-3,4- |
| | | carboxamide; hydro- | 1 H) 7.44 (d, J = 8.36 Hz, 1 H) | dimethylbenzoic acid, |
| | | chloride | 7.21 (t, J = 7.04 Hz, 1 H) 3.86 (m, | 2-chloronicotinic |
| | | | J = 6.20 Hz, 2 H) 3.62-3.69 (m, 2 | acid, and 2- |
| | | | H) 3.42-3.47 (m, 2 H) 2.99- | (pyrrolidin-1- |
| | | | 3.19 (m, 2 H) 2.58 (s, 3 H) 2.52 | yl)ethan-1-amine to |
| | | | (s, 3 H) 1.73-2.13 (m, 4 H). | give 21.2 mg, 83% |
| | | | | Yield as a yellow |
| | | | | solid. |
| 18 | 118 | 11-oxo-4-phenyl- | MS, ES⁺ m/z = 413.0, ¹H NMR | Method C (with |
| | | N-(2-(pyrrolidin-1- | (400 MHz, DMSO-d6) δ 10.65 | modified step 1 as |
| | | yl)ethyl)-11H- | (t, J = 6.0 Hz, 1H), 10.16-9.98 | follows: 1M HCl as |
| | | pyrido[2,1- | (m, 1H), 9.07 (dd, J = 7.2, 1.7 | solvent, heating to |
| | | b]quinazoline-6- | Hz, 1H), 8.64 (dd, J = 7.0, 1.7 | 110° C.) using 2- |
| | | carboxamide; hydro- | Hz, 1H), 8.41 (dd, J = 8.1, 1.5 | amino-[1,1′- |
| | | chloride | Hz, 1H), 7.96 (dd, J = 7.3, 1.5 | biphenyl]-3- |
| | | | Hz, 1H), 7.74-7.51 (m, 6H), | carboxamide, 2- |
| | | | 7.25 (t, J = 7.1 Hz, 1H), 3.45- | chloronicotinic acid, |
| | | | 3.27 (m, 4H), 2.95-2.79 (m, | and 2-(pyrrolidin-1- |
| | | | 4H), 2.05-1.89 (m, 2H), 1.89- | yl)ethan-1-amine to |
| | | | 1.74 (m, 2H). | give 52 mg, 77% |
| | | | | Yield as a yellow |
| | | | | solid. |
| 19 | 119 | 11-oxo-3-phenyl- | MS, ES⁺ m/z = 413.0, ¹H NMR | Method C using 3- |
| | | N-(2-(pyrrolidin-1- | (DMSO-d6, 500 MHz): δ (ppm) | amino-[1,1′- |
| | | yl)ethyl)-11H- | 11.16 (t, J = 6.0 Hz, 1H), 10.78 | bipheny1]-4- |
| | | pyrido[2,1- | (br s, 1H), 9.04 (dd, J = 7.3, 1.7 | carboxylic acid, - |
| | | b]quinazoline-6- | Hz, 1H), 8.65 (dd, J = 6.8, 1.7 | chloronicotinic acid, |
| | | carboxamide; hydro- | Hz, 1H), 8.49 (d, J = 1.7 Hz, 1H), | and 2-(pyrrolidin-1- |
| | | chloride | 8.39 (d, J = 8.6 Hz, 1H), 7.94- | yl)ethan-1-amine to |
| | | | 7.97 (m, 2H), 7.92 (dd, J = 8.4, | give 70.2 mg, 78% |
| | | | 1.8 Hz, 1H), 7.48-7.61 (m, 3H), | Yield as a pale pink |
| | | | 7.23 (t, J = 7.2 Hz, 1H), 3.91 (q, | solid. |
| | | | J = 6.1 Hz, 2H), 3.60-3.74 (m, | |
| | | | 2H), 3.46 (q, J = 6.0 Hz, 2H), | |
| | | | 2.95-3.18 (m, 2H), 1.72-2.11 (m, | |
| | | | 4H). | |

### Example 20: 4-methyl-11-oxo-N-(2-(Pyrrolidin-1-Yl)ethyl)-11H-pyrido[2,1-b]quinazoline-6-carboxamide;hydrochloride (Compound 117)

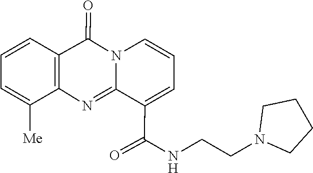 [SMILES: Cc1cccc2c(=O)n3cccc(C(=O)NCCN4CCCC4)c3nc12]

### Step 1: Preparation of 4-methyl-11-oxo-11H-pyrido[2,1-b]quinazoline-6-carboxylic acid

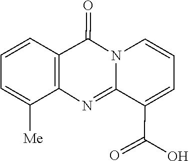 [SMILES: Cc1cccc2c(=O)n3cccc(C(=O)O)c3nc12]

In a vial, under inert atmosphere, to a suspension of 2-chloropyridine-3-carboxylic acid (517 mg, 3.28 mmol) and 2-amino-3-methylbenzoic acid (98%, 566 mg, 3.67 mmol) in a mixture of solvents DMF (2 mL)/Water (2 mL) was added sulfuric acid (98%, 2.0 mL, 36.8 mmol) dropwise at room temperature and the reaction mixture was stirred at 130° C. during 9 h. Sulfuric acid (98%, 2.0 mL, 36.8 mmol), water (1 mL) and DMF (1 mL) were added to the reaction mixture which was stirred at 110° C. during 16 h. The mixture was extracted with DCM. The combined organic layers were washed with brine, dried over sodium sulfate and dried in vacuum. The crude material was purified by Flash Chromatography on silica gel using a gradient of DCM to DCM 90/10 to 4-methyl-11-oxo-pyrido[2,1-b]quinazoline-6-carboxylic acid (125 mg, 3.7% Yield) as a yellow solid. MS, ES⁺ m/z=255.0, ¹H NMR (400 MHz, DMSO-d6) δ 9.08 (dd, J=7.2, 1.5 Hz, 1H), 8.68 (d, J=5.7 Hz, 1H), 8.23 (d, J=8.6 Hz, 1H), 7.93 (d, J=7.1 Hz, 1H), 7.64-7.39 (m, 4H), 7.31 (t, J=7.1 Hz, 1H), 7.18 (dd, J=21.5, 7.2 Hz, 3H), 6.51-6.43 (m, 2H), 2.61 (d, J=4.7 Hz, 2H), 2.23 (d, J=8.2 Hz, 1H), 2.10 (s, 6H).

### Step 2: Preparation of 4-methyl-11-oxo-N-(2-pyrrolidin-1-ylethyl)pyrido[2,1-b]quinazoline-6-carboxamide

The title compound was prepared by Method C steps 2-3 using 4-methyl-11-oxo-11H-pyrido[2,1-b]quinazoline-6-carboxylic acid (25%, 125 mg, 0.123 mmol), 2-(pyrrolidin-1-yl)ethan-1-amine pyrrolidine (98%, 0.20 mL, 1.54 mmol) to give (19 mg, 48% Yield) as a yellow solid. MS, ES⁺ m/z=351.0, ¹H NMR (DMSO-d6, 500 MHz): δ (ppm) 11.26 (t, J=6.0 Hz, 1H), 9.98 (br s, 1H), 9.05 (dd, J=7.1, 1.7 Hz, 1H), 8.65 (dd, J=6.8, 1.7 Hz, 1H), 8.21 (d, J=7.6 Hz, 1H), 7.90 (d, J=7.1 Hz, 1H), 7.47-7.57 (m, 1H), 7.25 (t, J=7.1 Hz, 1H), 3.86 (q, J=6.2 Hz, 2H), 3.60-3.69 (m, 2H), 3.45 (q, J=6.3 Hz, 2H), 3.01-3.19 (m, 2H), 2.69 (s, 3H), 1.71-2.18 (m, 4H), 1.04 (d, J=5.9 Hz, 1H).

### Example 21: 2,2-difluoro-6-oxo-N-(2-(pyrrolidin-1-yl)ethyl)-6H-[1,3]dioxolo[4,5-h]pyrido[2,1-b]quinazoline-11-carboxamide;hydrochloride (Compound 121)

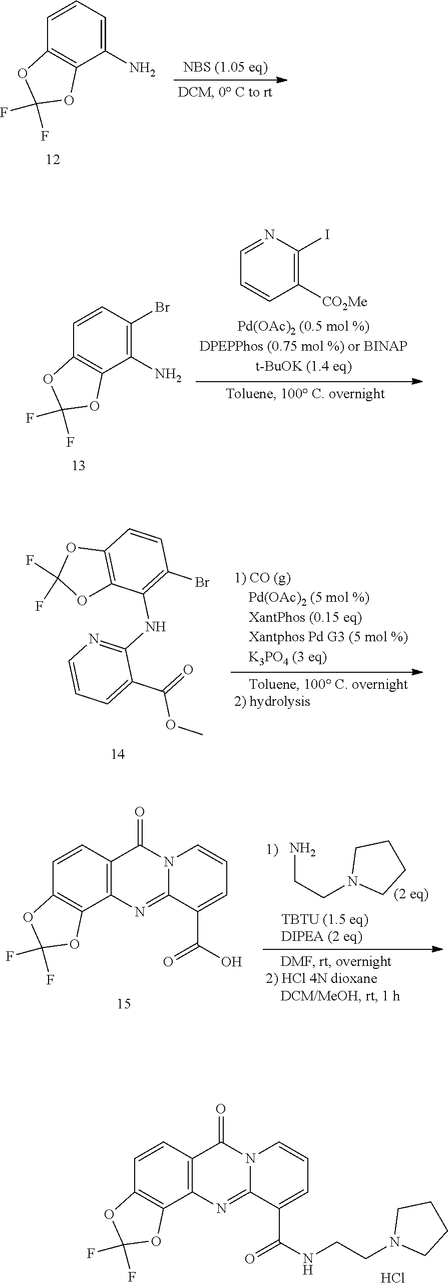

### Step 1: Preparation of 5-bromo-2,2-difluorobenzo[d][1,3]dioxol-4-amine

To a stirred solution of 2,2-difluoro-1,3-benzodioxol-4-amine (96%, 2.50 g, 13.9 mmol) in anhydrous DCM (30 ml) at 0° C. was added 1-bromopyrrolidine-2,5-dione (2.59 g, 14.6 mmol) portionwise. The solution was stirred at 0° C. for 30 min then warmed to room temperature and was stirred 1 h more. The reaction mixture was quenched with 1 M sodium thiosulfate solution and diluted with deionized water, extracted with dichloromethane. The combined organic layers were dried over sodium sulfate, filtered, and concentrated in vacuum. The crude material was purified via automated normal phase liquid chromatography on a 90 g SI60 15-40 μM column with Heptane to Heptane75/25AcOEt to give 5-bromo-2,2-difluoro-1,3-benzodioxol-4-amine (2.53 g, 72% Yield) as an off-white solid. ¹H NMR (500 MHz, DMSO-d₆) δ 7.23 (d, J=8.6 Hz, 1H), 6.56 (d, J=8.6 Hz, 1H), 5.80 (s, 2H).

### Step 2: Preparation of methyl 2-((5-bromo-2,2-difluorobenzo[d][1,3]dioxol-4-yl)amino)nicotinate

A 2-5 ml microwave vial was charged with methyl 2-chloropyridine-3-carboxylate (98%, 250 mg, 1.43 mmol) in anhydrous Toluene (3 ml). The mixture was purged three times with Argon. Then 5-bromo-2,2-difluoro-1,3-benzodioxol-4-amine (90%, 333 mg, 1.19 mmol), diacetoxypalladium (11 mg, 0.0476 mmol), [1-(2-diphenylphosphanyl-1-naphthyl)-2-naphthyl]-diphenyl-phosphane (44 mg, 0.0714 mmol) and cesium carbonate (543 mg, 1.67 mmol) were successively added. The mixture was purged again three times with Argon before the vial was sealed. The reaction mixture was heated 1.5 h at 130° C. using microwaves, then quenched with water and diluted with DCM. The layers were separated and aqueous layer was extracted with DCM. The organic layers were combined, washed with brine, dried over sodium sulfate, filtered and concentrated in vacuum. The crude material was purified via automated normal phase liquid chromatography on a 30 g SI60 15-40 μM column with Heptane to Heptane 75/25 AcOEt to give methyl 2-[(5-bromo-2,2-difluoro-1,3-benzodioxol-4-yl)amino]pyridine-3-carboxylate (130 mg, 28% Yield) as an off-white solid. ¹H NMR (400 MHz, DMSO-d6) δ 9.57 (s, 1H), 8.35-8.25 (m, 2H), 7.59 (d, J=8.7 Hz, 1H), 7.29 (d, J=8.7 Hz, 1H), 6.97 (dd, J=7.8, 4.8 Hz, 1H), 3.93 (s, 3H). MS [M+1]=389

### Step 3: Preparation of methyl 2,2-difluoro-6-oxo-6H-[1,3]dioxolo[4,5-h]pyrido[2,1-b]quinazoline-11-carboxylate

In a 2-5 ml microwave vial was dissolved methyl 2-[(5-bromo-2,2-difluoro-1,3-benzodioxol-4-yl)amino]pyridine-3-carboxylate (130 mg, 0.336 mmol) in anhydrous Toluene (3 ml). The mixture was purged 3 times with argon then diacetoxypalladium (7.5 mg, 0.0331 mmol), (5-diphenylphosphanyl-9,9-dimethyl-xanthen-4-yl)-diphenyl-phosphane (29 mg, 0.050 mmol), XantPhos Pd G3(95%, 17 mg, 0.0170 mmol) and tripotassium;phosphate (217 mg, 1.01 mmol) were added. The mixture was purged again 3 times before adding hexakis(oxomethylidene)molybdenum (133 mg, 0.504 mmol). The vial was sealed and stirred at 115° C. overnight. The mixture was filtered, washed with EtOAc and dried in vacuum. The crude material was purified via automated normal phase liquid chromatography on a 30 g SI60 15-40 μM column with Heptane to Heptane 75/25AcOEt to give methyl 13,13-difluoro-2-oxo-12,14-dioxa-3,9-diazatetracyclo[8.7.0.03,8.011,15]heptadeca-1(10),4,6,8,11(15),16-hexaene-7-carboxylate (36 mg, 25% Yield) as a yellow gum. ¹H NMR (400 MHz, DMSO-d6) δ 8.93 (dd, J=7.3, 1.6 Hz, 1H), 8.24 (d, J=8.8 Hz, 1H), 8.10 (dd, J=6.8, 1.6 Hz, 1H), 7.64 (d, J=8.8 Hz, 1H), 7.20-7.12 (m, 1H), 3.93 (s, 3H). MS [M+1]=335.

### Step 4: Preparation of 2,2-difluoro-6-oxo-6H-[1,3]dioxolo[4,5-h]pyrido[2,1-b]quinazoline-11-carboxylic acid

In a 25 ml round bottom flask was solubilized methyl 13,13-difluoro-2-oxo-12,14-dioxa-3,9-diazatetracyclo[8.7.0.03,8.011,15]heptadeca-1(10),4,6,8,11(15),16-hexaene-7-carboxylate (77%, 36 mg, 0.0829 mmol) in MeOH (1 ml). Then a solution of 1 M hydrogen chloride (1.0 mL, 1.01 mmol) in water was added. The mixture was heated and stirred 5 h at reflux (100° C.). The mixture was diluted with water and extracted with DCM. The organic layers were combined, washed with brine, dried over sodium sulfate, filtered and concentrated in vacuum to give 13,13-difluoro-2-oxo-12,14-dioxa-3,9-diazatetracyclo[8.7.0.03,8.011,15]heptadeca-1(10),4,6,8,11(15),16-hexaene-7-carboxylic acid (17 mg, 46% Yield) as a yellow solid. MS [M+1]=321. Crude material was used without further purification in the coupling step.

### Step 5: Preparation of 2,2-difluoro-6-oxo-N-(2-(pyrrolidin-1-yl)ethyl)-6H-[1,3]dioxolo[4,5-h]pyrido[2,1-b]quinazoline-11-carboxamide

In a 2-5 ml vial was added 13,13-difluoro-2-oxo-12,14-dioxa-3,9-diazatetracyclo[8.7.0.03,8.011,15]heptadeca-1(10),4,6,8,11(15),16-hexaene-7-carboxylic acid (73%, 17 mg, 0.0388 mmol) in anhydrous DMF (1 ml). Then N-(2-aminoethyl)pyrrolidine (8.9 mg, 0.0775), N-ethyl-N-isopropyl-propan-2-amine (0.020 ml, 0.116 mmol) and [benzotriazol-1-yloxy(dimethylamino)methylene]-dimethyl-ammonium;tetrafluoroborate (17 mg, 0.0543 mmol) were successively added. The mixture was stirred 6 h at room temperature. The mixture was quenched with a solution of sodium hydrogen carbonate sat. then extracted with DCM. The organics layers were combined, washed with brine, dried over sodium sulfate, filtered and concentrated in vacuum. The crude material was purified via automated normal phase liquid chromatography on a 15 g SI60 15-40 μM column with DCM to DCM 90/10 MeOH to give 13,13-difluoro-2-oxo-N-(2-pyrrolidin-1-ylethyl)-12,14-dioxa-3,9-diazatetracyclo[8.7.0.03,8.011,15]heptadeca-1(10),4,6,8,11(15),16-hexaene-7-carboxamide (7 mg, 40% Yield) as a yellow solid. MS [M+1]=417.

### Step 6: Preparation of 2,2-difluoro-6-oxo-N-(2-(pyrrolidin-1-yl)ethyl)-6H-[1,3]dioxolo[4,5-h]pyrido[2,1-b]quinazoline-11-carboxamide;hydrochloride

To a 100 ml round bottom flask was dissolved 13,13-difluoro-2-oxo-N-(2-pyrrolidin-1-ylethyl)-12,14-dioxa-3,9-diazatetracyclo[8.7.0.03,8.011,15]heptadeca-1(10),4,6,8,11(15),16-hexaene-7-carboxamide (92%, 7.1 mg, 0.0156 mmol) in a DCM/MeOH 1/1 mixture (1 ml). A solution of 4 M 1,4-dioxane hydrochloride (12 uL, 0.0468 mmol) was added dropwise and the reaction mixture was stirred 1 h at room temperature. Diethyl ether was then added until precipitation of a yellow solid. The solid was filtered and dried in vacuum to give 13,13-difluoro-2-oxo-N-(2-pyrrolidin-1-ylethyl)-12,14-dioxa-3,9-diazatetracyclo[8.7.0.03,8.011,15]heptadeca-1(10),4,6,8,11(15),16-hexaene-7-carboxamide;hydrochloride (7 mg, 99% Yield) as a yellow solid. ¹H NMR (600 MHz, DMSO-d₆) δ ppm 1.75-2.13 (m, 4H) 3.02-3.22 (m, 2H) 3.35-3.51 (m, 2H) 3.57-3.73 (m, 2H) 3.73-3.90 (m, 2H) 7.29 (t, J=7.12 Hz, 1H) 7.68 (d, J=8.80 Hz, 1H) 8.28 (d, J=8.80 Hz, 1H) 8.60 (br d, J=6.75 Hz, 1H) 9.06 (dd, J=7.19, 1.61 Hz, 1H) 9.53-9.96 (m, 1H) 10.43 (br s, 1H). MS [M+1]=417.

Cell culture: A375 cells (CRL-1619, ATCC) were grown in DMEM (GIBCO) supplemented with 10% fetal bovine serum (GIBCO) in 5% CO2 at 37° C.

Cell treatment and Imaging: 18 hours post-seeding in cell carrier black 384-well plates (PerkinElmer) at 15 000 cells/well, A375 cells were treated for 4 hours with a positive control or test compounds (9 serial semi-log dilutions, top concentration 10 μM). Upon treatment A375 cells were (i) fixed for 15 min at RT with 4% of paraformaldehyde, (ii) permeabilized for 20 min at RT with 0.1-0.5% of NP-40, (iii) incubated with primary antibodies at 4° C.: anti-RPA194 (1:1000-1:1250 dilution, sc-48385, Santa Cruz Biotechnology) and anti-gH2Ax (1:5000 dilution, 81299, Abcam or 1:4000, JBW302, Millipore) (iv) incubated for 1 h at RT with Hoechst (1:5000 dilution, H3570, ThermoFisher) and secondary antibodies: anti-mouse-AlexaFluor488 (1:2000 dilution, A11001, ThermoFisher) and anti-rabbit-AlexaFluor647 (1:2000 dilution, A21443, ThermoFisher) or goat anti-mouse AlexaFluor-594 (1:1000 dilution). Images were acquired on an Operetta CLS High-Content Analysis System (PerkinElmer) (40× objective; 9 fields/well) and processed using the Columbus Image Analysis System (PerkinElmer) or by using Molecular Devices ImageXpress Micro XLS High Content Imager and MetaXpress high-content acquisition and analysis software.

Cell viability assay: 18 hours post-seeding in white 384-Viewplate (6007480, PerkinElmer) at 800 cells/well, or Costar black 96-well plate; (#3603) at 14,000 cells/well, A375 cells were treated for 72 hours with positive controls or test compounds (9 serial semi-log dilutions, top concentration 10 μM). Following treatment, A375 cells were incubated for 10 min at RT with the CellTiter-Glo reagent (G7571, Promega) or CellTiter-Blue® Reagent (G8081, Promega). Measurement of the luminescence signal was performed on an Ensight (PerkinElmer) or Victor Nivo™ Multimode Plate reader.

As the data herein indicate, a broad variety of compounds of Formula (I) were found to effectively destruct RPA194 and inhibit RNA polymerase I (Pol I) at low concentrations. pIC₅₀ values for exemplary compounds of Formula (I) (see Table C1 compound names and structures) are provided in Table B1 below. Any compound with pIC₅₀ superior or equal to 5.02 in this assay, as described above, is deemed a Pol I inhibitor. In the table below, (+) is associated with a pIC₅₀ 5.02 to 5.8; (++) with pIC₅₀ 5.8 to 6.3; and plus sing (+++) with a pIC50 above 6.3.

TABLE B1

| | Compound | Activity |
|---|---|---|
| Example- | No. | Range |
| 1 | 107 | +++ |
| 2 | 106 | + |
| 3 | 105 | ++ |
| 4 | 102 | + |
| 5 | 104 | + |
| 6 | 103 | ++ |
| 7 | 101 | + |
| 8 | 113 | + |
| 9 | 116 | + |
| 10 | 114 | + |
| 11 | 108 | ++ |
| 12 | 109 | ++ |
| 13 | 110 | ++ |
| 14 | 111 | + |
| 15 | 112 | ++ |
| 16 | 115 | +++ |
| 17 | 120 | +++ |
| 18 | 118 | + |
| 19 | 119 | + |
| 20 | 117 | ++ |
| 21 | 121 | + |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A compound of Formula (I): 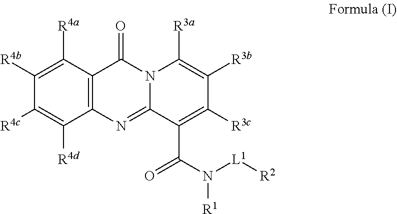
or a pharmaceutically acceptable salt thereof, wherein:
R¹ is H or C₁₋₃ alkyl;
R² is selected from the group consisting of:
(a) —NR⁶R⁷, wherein R⁶ and R⁷ are independently selected from the group consisting of: H and C₁₋₆ alkyl which is optionally substituted with from 1-6 R^{a}; and
(b) -heterocyclyl including from 4-12 ring atoms, wherein from 1-3 ring atoms are ring heteroatoms each independently selected from the group consisting of N, NH, N(R^{d}), O, and S(O)₀₋₂, wherein the heterocyclyl is optionally substituted with from 1-6 R^{b};
L¹ is a bond or C₁₋₆ alkylene which is optionally substituted with from 1-6 R^{c}, provided that when L¹ is a bond, then R² is heterocyclyl that is attached to L¹ via a ring carbon atom;
R^{3a}, R^{3b}, R^{3c}, R^{4a}, R^{4b}, R^{4c}, and R^{4d} are each independently selected from the group consisting of: H, halo, C₁₋₆ alkyl, C₁₋₆ haloalkyl, C₁₋₆ alkoxy, C₁₋₆ haloalkoxy, OH, C₃₋₆ cycloalkyl, and C₆₋₁₀ aryl; or
two of the variables R^{4a}, R^{4b}, R^{4c}, and R^{4d} on adjacent ring carbon atoms, taken together with the ring carbon atoms to which each is attached, forms a partially unsaturated ring including from 4-8 ring atoms, wherein from 0-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b};
each occurrence of R^{a} and R^{c} is independently selected from the group consisting of: —OH; -halo; —NR′R″; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently selected from the group consisting of: C₁₋₆ alkyl optionally substituted with —OH, C₁₋₄ alkoxy, or C₁₋₄ haloalkoxy; C₁₋₆ haloalkyl; oxo; —OH; -halo; —NR′R″; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{d} is independently C₁₋₆ alkyl; —C(O)(C₁₋₄ alkyl); or —C(O)O(C₁₋₄ alkyl); and
each occurrence of R′ and R″ is independently H or C₁₋₃ alkyl.

2. The compound of claim 1, wherein R^{4b} and R^{4c} taken together with the ring carbon atoms to which each is attached, forms a partially unsaturated ring including from 4-8 ring atoms, wherein from 0-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b}.

3. The compound of claims 1 or 2, wherein R^{4b} and R^{4c} taken together with the ring carbon atoms to which each is attached, forms a partially unsaturated ring including from 4-6 ring atoms, wherein from 1-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b}.

4. The compound of any one of claims 1-3, wherein R^{4b} and R^{4c} taken together with the ring carbon atoms to which each is attached forms:

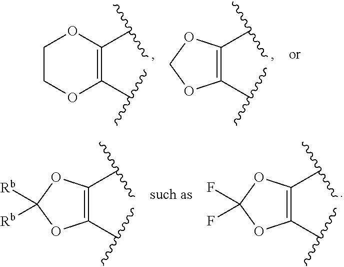

5. The compound of claims 1 or 2, wherein R^{4b} and R^{4c} taken together with the ring carbon atoms to which each is attached, forms a partially unsaturated ring including from 4-8 ring atoms, wherein the partially unsaturated ring does not include a ring heteroatom, and wherein the partially unsaturated ring is optionally substituted with from 1-3 R^{b}, such as wherein R^{4b} and R^{4c} taken together with the carbon atoms to which each is attached forms

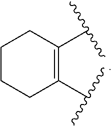 [SMILES: *C1=C(*)CCCC1]

6. The compound of claim 1, wherein R^{4b} and R^{4c} are independently C₁₋₆ alkyl, C₁₋₆ haloalkyl, C₃₋₆ cycloalkyl, or C₆₋₁₀ aryl.

7. The compound of claims 1 or 6, wherein R^{4b} and R^{4c} are independently selected C₁₋₃ alkyl, such as methyl.

8. The compound of any one of claims 1-7, wherein R^{4a} and R^{4d} are H.

9. The compound of claim 1, wherein R^{4c} and R^{4d} taken together with the ring carbon atoms to which each is attached, forms a partially unsaturated ring including from 4-8 ring atoms, wherein from 0-2 ring atoms are ring heteroatoms each independently selected from the group consisting of: O, N, N(H), N(R^{d}), and S(O)₀₋₂, and wherein the partially unsaturated ring is optionally substituted with from 1-3 R^{b}, such as wherein R^{4c} and R^{4d} taken together with the carbon atoms to which each is attached forms

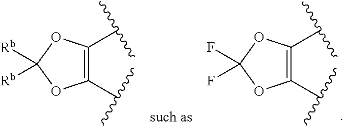

10. The compound of claim 1, wherein R^{4c} and R^{4d} are independently C₁₋₆ alkyl, C₁₋₆ haloalkyl, C₃₋₆ cycloalkyl, or C₆₋₁₀ aryl.

11. The compound of claims 1 or 10, wherein R^{4c} and R^{4d} are independently selected C₁₋₃ alkyl, such as methyl.

12. The compound of claim 1, wherein R^{4c} is C₁₋₆ alkyl, C₁₋₆ haloalkyl, C₃₋₆ cycloalkyl, or C₆₋₁₀ aryl; and R^{4d} is H.

13. The compound of claims 1 or 12, wherein R^{4c} is C₁₋₃ alkyl, such as methyl; and R^{4d} is H.

14. The compound of claims 1 or 12, wherein R^{4c} is phenyl; and R^{4d} is H.

15. The compound of claim 1, wherein R^{4d} is C₁₋₆ alkyl, C₁₋₆ haloalkyl, C₃₋₆ cycloalkyl, or C₆₋₁₀ aryl; and R^{4c} is H.

16. The compound of claims 1 or 15, wherein R^{4d} is C₁₋₃ alkyl, such as methyl; and R^{4c} is H.

17. The compound of claims 1 or 15, wherein R^{4d} is phenyl; and R^{4c} is H.

18. The compound of any one of claims 9-17, wherein R⁴¹ and R^{4b} are H.

19. The compound of any one of claims 1-18, wherein L¹ is C₁₋₆ alkylene optionally substituted with from 1-6 R^{c}.

20. The compound of any one of claims 1-19, wherein L¹ is a linear C₁₋₄ alkylene, optionally substituted with from 1-6 R^{c}.

21. The compound of any one of claims 1-20, wherein L¹ is unsubstituted —CH₂CH₂—.

22. The compound of any one of claims 1-19, wherein L¹ is a branched C₃₋₄ alkylene optionally substituted with from 1-6 R^{c}, such as wherein L¹ is 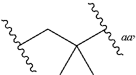
wherein aa is the point of attachment to R².

23. The compound of any one of claims 1-22, wherein R² is NR⁶R⁷.

24. The compound of any one of claims 1-23, wherein R² is selected from the group consisting of: NH(C₁₋₃ alkyl) and N(C₁₋₃ alkyl)₂, such as wherein R² is —NMe₂.

25. The compound of any one of claims 1-22, wherein R² is heterocyclyl including from 4-12 ring atoms, wherein from 1-3 ring atoms are ring heteroatoms each independently selected from the group consisting of N, NH, N(R^{d}), O, and S(O)₀₋₂, wherein the heterocyclyl is optionally substituted with from 1-6 R^{b}.

26. The compound of any one of claims 1-22 or 25, wherein R² is heterocyclyl including from 4-8 such as 4-6 ring atoms, wherein from 1-2 ring atoms are ring heteroatoms each independently selected from the group consisting of N, NH, N(R^{d}), O, and S(O)₀₋₂, wherein the heterocyclyl is optionally substituted with from 1-6 R^{b}.

27. The compound of any one of claims 1-22 or 25-26, wherein R² is selected from the group consisting of:

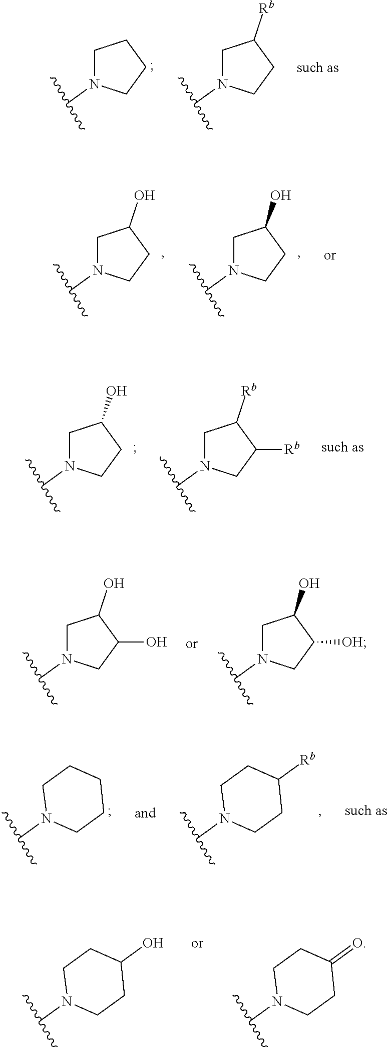

28. The compound of any one of claims 1-27, wherein R¹ is H.

29. The compound of any one of claims 1-28, wherein R^{3a}, R^{3b}, and R^{3c} are H.

30. The compound of any one of claims 1-29, wherein the compound is selected from the group consisting of the compounds in Table C1, or a pharmaceutically acceptable salt thereof.

31. A pharmaceutical composition comprising a compound of any of claims 1-30, and a pharmaceutically acceptable carrier.

32. A method for activating upstream p53 pathways in a mammalian cell, wherein the method comprises contacting a cell or population of cells with a compound as claimed in any one of claims 1-30, or a pharmaceutically acceptable salt thereof, or a pharmaceutical composition as claimed in claim 31.

33. A method for modulating RNA Pol I activity in a mammalian cell, wherein the method comprises contacting a cell or population of cells with a compound as claimed in any one of claims 1-30, or a pharmaceutically acceptable salt thereof, or a pharmaceutical composition as claimed in claim 31.

34. A method for treating cancer in a subject, wherein the method comprises administering to the subject a compound as claimed in any one of claims 1-30, or a pharmaceutically acceptable salt thereof, or a pharmaceutical composition as claimed in claim 31.

35. A method for treating an autoimmune disease or disorder in a subject, wherein the method comprises administering to the subject a compound as claimed in any one of claims 1-30, or a pharmaceutically acceptable salt thereof, or a pharmaceutical composition as claimed in claim 31.

36. A method for treating a condition associated with inflammation or pain in a subject, wherein the method comprises administering to the subject a compound as claimed in any one of claims 1-30, or a pharmaceutically acceptable salt thereof, or a pharmaceutical composition as claimed in claim 31.

37. The method of any one of claims 34-36, further comprising administering to the subject at least one additional therapeutic agent.
